(19) United States
(12) Patent Application Publication
(10) Pub. No.: US 2026/0306434 A1
(43) Pub. Date: Oct. 8, 2026

(54) VERY LONG CHAIN FATTY ACIDS FOR USE IN MINERALIZATION

(71) Applicant: Epax Norway AS, Ålesund (NO)

(72) Inventors: Martina TORRISSEN, Oslo (NO); Bente RUYTER, Ås (NO); Elisabeth YTTERBORG, Oslo (NO); Harald SVENSEN, Ellingsøy (NO); Derek TOBIN, Kolsås (NO); Iren Merete Skjåstad STOKNES, Ålesund (NO)

(73) Assignee: Epax Norway AS, Ålesund (NO)

(21) Appl. No.: 19/476,317

(22) PCT Filed: Apr. 26, 2024
(86) PCT No.: PCT/NO2024/050102

§ 371 Date: Oct. 17, 2025

(30) Foreign Application Priority Data
Apr. 26, 2023 (NO) 20230466

(51) Int. Cl. A61K 31/202; A61P 19/08

(52) U.S. Cl. CPC A61K 31/202; A61P 19/08

(57) ABSTRACT

The invention relates to compositions of very long chain fatty acids (VLCFAs) and to the biological effects of these fatty acids particularly on tissues, skin and bone. Particularly, use of VLCFAs has been found to affect uptake of important minerals for bone mineralization, indicating that VLCFAs stimulate to faster cell migration, cell proliferation and mineralization of bone cells. VLCFAs have hence been found to strengthen skin tissue and increase mineral uptake in skin and bone after supplementation.

### TECHNICAL FIELD OF THE INVENTION

The present invention relates to methods and use of compositions of very long chain fatty acids (VLCFAs), and to the biological effects of these fatty acids particularly on tissues, skin and bone. The invention further relates to compositions that can be utilised to increase mineralization, such as to improve mineralization in skin tissue or bone, wherein the composition comprises VLCFAs. Particularly, use of VLCFAs has been found to affect uptake of important minerals for bone mineralization, indicating that VLCFAs stimulate to faster cell migration, cell proliferation and mineralization of bone cells. VLCFAs have hence been found to strengthen skin tissue and increase mineral uptake in skin and bone after supplementation. Hence, it is suggested that VLCFAs could be useful in fracture prevention and/or bone fragility treatment.

### BACKGROUND OF THE INVENTION

Polyunsaturated fatty acids (PUFAs) play major roles in skin barrier function and integrity and several in vivo studies have demonstrated that consumption of PUFAs improves skin barrier function in the presence of essential fatty acid deficiency or skin disease. The anti-inflammatory properties of omega-3 (n-3) fatty acids from fish oils are well established. Studies have shown greater epithelialisation of blisters in groups receiving n-3 fatty acid supplementation compared with controls, suggesting improved wound healing, in addition to reduced wound infections in humans. A possible hypothesis is that marine n-3 fatty acids increase the migration of cells to the wound bed by inducing specialised pro-resolving metabolic pathways, resulting in lower levels of inflammation in a similar manner to that of the downstream effects seen with non-steroidal anti-inflammatory drugs (Patel M, li JJCWCMR (2019) Fish skin acellular dermal matrix: potential in the treatment of chronic wounds. 6, 59-70).

Several studies have indicated critical tissue-specific roles of very long chain fatty acids (VLCFAs), which are defined as fatty acids with a chain length of ≥24 carbon atoms, including in skin tissue (Vasireddy V, Uchida Y, Salem N, Jr. et al. (2007) Loss of functional ELOVL4 depletes very long-chain fatty acids (>or =C28) and the unique omega-O-acylceramides in skin leading to neonatal death. Human molecular genetics 16, 471-482). This stems from the finding that mutations in the elongase of very long chain fatty acids-4 (ELOVL4) gene, that encodes the ELOVL4 protein, which is responsible for mediating the biosynthesis of these fatty acids, is associated with several tissue-specific disorders. VLCFAs are generally not obtained via the diet and thus mutations in ELOVL4 results in a lack of these fatty acids in tissues where they are normally present, and consequently disorders of different magnitudes, indicating their importance. Studies in skin tissue revealed that loss of functional ELOVL4 protein is associated with skin disorders, defective skin permeability barrier, and has been shown to be lethal during early development (Vasireddy et al). The severity of these conditions indicates that VLCFAs play an essential role, however, their specific function(s) and products remain to be established. The applicant has previously demonstrated clear deposition of n-3 very long chain polyunsaturated fatty acids (VLCPUFAs, or VLC-PUFAs) in skin tissue after dietary supplementation in Atlantic salmon (Salmo salar) and mice, see WO2020/242322. This is an interesting finding, as the importance of VLCFAs in skin has mostly been examined in relation to very-long-chain saturated fatty acids (VLC-SFAs), and thus the role of VLCPUFAs remains unclear. These findings indicate that VLCPUFAs also play a role in the maintenance of normal skin tissue homeostasis. Therefore, the applicant has invested further to understand the effect of incorporation of VLCFAs in skin and their role in skin functions, and other tissue.

### BRIEF SUMMARY OF THE INVENTION

The present invention relates to compositions that can be utilised to increase mineralization, such as to improve mineralization in skin tissue or bone, wherein the composition comprises very long chain fatty acids. Particularly, use of VLCFAs has been found to affect uptake of important minerals for bone mineralization, indicating that VLCFAs stimulate to faster cell migration, cell proliferation and mineralization of bone cells. VLCFAs have hence been found to strengthen skin tissue and increase mineral uptake in skin and bone after administration.

Accordingly, the invention relates to compositions comprising very long chain fatty acids having a chain length of 24 carbon atoms or more, for one or more of
increase mineralization in bone or tissue;
Increase bone density, such as bone mineral density;
improve cell proliferation and cell growth;
improve cell migration.

In one aspect, the invention provides the use of a very long chain fatty acid (VLCFA) composition comprising a mixture of at least two very long chain polyunsaturated fatty acids (VLCPUFAs), wherein the VLCPUFAs have a chain length of 24 carbon atoms or more, in a method for increasing the amount of one or more of Phosphorus (P), Calcium (Ca) or Magnesium (Mg) in skin or bone tissue of a subject, the method comprising administering the VLCFA composition to the subject.

The VLCFAs and compositions comprising these can be included in, or used as, pharmaceuticals, supplements, food, or feed, as further detailed herein.

Furthermore, VLCFAs, or compositions comprising VLCFAs, are for use in fracture prevention and/or bone fragility treatment, such as for maintaining or improving bone density. In one aspect, the invention provides compositions for use in prevention or treatment of bone fractures or bone fragility, the composition comprising a mixture of at least two very long chain polyunsaturated fatty acids having a chain length of 24 carbon or more, wherein the composition is administered to a subject.

### BRIEF DESCRIPTION OF THE DRAWINGS

FIG. 1 Shows the VLCPUFA composition (μg/g tissue) in the phospholipid fraction from the skin of salmon fed a diet with different levels of VLCPUFA (VLC-Conc1), 0, 2.5, 5.0, 7.5, 10.0% of VLC-Conc1, reference is made Example 1.

FIG. 2 shows histology data of salmon skin from fish fed a diet with different levels (%) of VLCPUFA (VLC-Conc1), reference is made Example 1. FIG. 2(a): Histological analysis of epidermis thickness, dermis thickness and mucus cell count per 100 μm (n 15 per group), at day 18 and day 28, respectively. Bars with different letters are significantly different (P<0-05). FIG. 2(b): Histological images of skin samples stained with Von Kassa. Left image shows diet group 0, day 18, with light colouring of scales, right image shows diet group 10, day 18 with darker colouring of scales. FIG. 2(c): Histological images of skin samples stained with H&E, for diet groups 0, 7-5 and 10, at day 18 and 28. Small letters denote identified structures in the skin as follows: ed, epidermis; sc, scale; mu, mucus cell; dm, dermis; at, adipose tissue; is, immature scale; ms, mature scale; mc, muscle. (Von Kassa and H&E staining, 200× and 300× magnifications).

FIG. 3 shows the VLCPUFA composition of the whole-body homogenate of Atlantic salmon fed diets with different fatty acid compositions, diets with 0, 2.5, 5, 7.5, and 10% VLCPUFAs of total fatty acids, respectively, reference is made to Example 3. Ctr, Control; L-VLC, Low-VLC; I-VLC, Intermediate-VLC; MH-VLC, Medium-High-VLC; H-VLC, High-VLC.

FIG. 4 shows X-ray CT analyses results, as body mineral density (BMD), of Atlantic salmon fed different diets. Ctr, Control; L-VLC, Low VLC; I-VLC, Intermediate VLC; MH-VLC, Medium-High VLC; H-VLC, High-VLC. Significance was set at P<0.05. The letters a-c denotes significant differences between the groups.

### DETAILED DESCRIPTION OF THE INVENTION

Biologically active polyunsaturated fatty acids (PUFAs), including omega-3 PUFAs, are not limited to the long chain fatty acids such as EPA and DHA. WO2016/182452 of Epax Norway AS discloses a method for producing compositions of very long chain polyunsaturated fatty acids (VLCPUFAs), specifically very long chain omega-3 fatty acids (VLCn3s), as well as compositions comprising high concentrations of such VLCn3s, from natural oils. WO2016/182452 further discloses that there is only a small amount of the VLCn3s in natural oils like fish oils, and explains why these and other very long chain fatty acids are substantially removed during production of traditional marine omega-3 concentrates, where the aim is to up-concentrate omega-3-fatty acids with chain length C20-C22.

Very long chain fatty acids, denoted VLCFAs or VLC-FAs herein, have a chain length of ≥24 carbon atoms and are generally not provided through dietary sources. They are synthesized in tissues expressing the enzyme responsible for their condensation reaction (Elongase of Very Long Chain Fatty Acids-4, ELOVL4), and emerging evidence suggest they play critical important roles in these tissues, which include retina, skin, testis, and brain. After identifying VLCFAs in fish oil and developing a method for concentrating very long chain polyunsaturated fatty acids (denoted VLCPUFAs or VLC-PUFAs herein) in kg scale, feeding trials have been conducted to investigate their biological effects in vivo.

The invention is based on several studies conducted, including two studies feeding Atlantic salmon with a feed comprising different levels of n-3 VLCPUFAs. The results demonstrate promising effects on development and maturation of skin tissue with increasing levels of n-3 VLCPUFAs, possibly providing a more robust skin at an earlier life stage. Additionally, the scales of the salmon showed increased mineralization with higher levels of n-3 VLCPUFAs in their feed, having analyzed mineral composition of skin and bone, as well as histological evaluations of H&E and Von Kossa stained skin samples demonstrating increased mineralization of scales. The results demonstrate increased uptake of several important minerals for bone mineralization, with increasing levels of n-3 VLCPUFAs in the feed. Furthermore, as there are many similarities between teleost scales and mammalian bone, further investigations have been made indicating that VLCPUFAs stimulate to faster cell migration and mineralization of human osteoblasts in vitro, and early data on proliferation and mineralization markers show interesting results. VLCFAs have hence been found to strengthen skin tissue and increase mineral uptake in skin and bone after supplementation.

The invention hence relates to very long chain fatty acids (VLCFAs), compositions comprising VLCFAs, and to the use of such fatty acids and compositions in improving mineralization in bone or tissue. Equally, the invention relates to methods and use of concentrates of VLCFAs to achieve a higher content of minerals in skin and bone cells as well as to increase the growth (proliferation and/or migration) of such cells.

The applicant's studies support that administration of VLCFAs contribute to increased mineralization in the skin and bones. Hence, VLCFA administration can contribute to important minerals entering bone cells and ensuring the renewal and strengthening of bone tissue. This is relevant in relation to bone fragility, healing of bone fractures and other damages to bone substance, or in prevention of deformities. Furthermore, VLCFAs contribute to increased expression of genes that are important for the proliferation of skin and bone cells. Accordingly, using VLCFAs a faster healing of injuries to the skin and bones can be achieved, faster improvement after surgery or insertion of implants may be achieved, or VLCFAs could be useful in fracture prevention and/or bone fragility treatment.

Studies conducted by the applicant and partners are summarized below together with key findings. Some of these are provided in detail in the Example section. While several earlier studies have shown that PUFAs have beneficial effects in wound healing assays, to our knowledge, our findings are the first to demonstrate the beneficial effects of VLCPUFAs in wound healing, cell migration, cell proliferation and mineralization.

The present studies contribute to the scarce literature on VLCPUFAs and highlights their potential effects on skin, skin health and mineralization, in both human and animal models. It is reasonable to assume that these unique fatty acids, although low in abundance, have some essential effects in the different organs and tissues in which they normally appear, considering the pathology observed in relation to ELOVL4 mutations. Based on the in vivo and in vitro observations presented herein, with similar findings in both human and fish, it is supported that VLCFAs have positive effects on skin tissue development, function, and integrity, as well as on mineral uptake and ensuring the renewal and strengthening of bone tissue.

Use of VLCFAs, and compositions comprising VLCFAs, may affect any one or more of:
improve mineralization in bone or tissue; increase uptake of minerals, e.g. P, Ca, Mg, e.g. in bone cells (osteoblasts);
Increase bone density, such as bone mineral density;
improve cell proliferation and cell growth;
improve, or enhance speed of, cell migration.

Accordingly, the invention provides the use of a VLCFA composition typically comprising a mixture of VLCPUFAs, in a method for increasing the amount of one or more of Phosphorus (P), Calcium (Ca) or Magnesium (Mg) in skin or bone tissue of a subject, the method comprising administering the VLCFA composition to the subject.

Accordingly, it is also foreseen that VLCFA compositions could be useful in new therapies for fracture prevention and/or bone fragility treatment, including in treatment or prevention of osteoporosis. Furthermore, the VLCFAs could be useful in healing of bone fractures and other damages to bone substance, or in prevention of deformities.

In one aspect, the invention relates to a composition comprising VLCFAs, for use in prevention or treatment of bone fractures or bone fragility, wherein the composition is administered to a subject. In one embodiment, such treatment is for maintaining or improving the bone density of the subject, such as the bone mineral density. The treatment may encompass therapeutic treatment. In an equal aspect, the invention provides a method of treating or preventing bone fragility of a subject, wherein the method comprising administering the VLCFA composition to the subject. The VLCFA composition comprises very long chain fatty acids having a chain length of 24 carbon or more, as further detailed below. In an equal aspect, the invention provides use of VLCFA composition as disclosed for the preparation of a medicament for prevention or treatment of bone fractures or bone fragility, such as of osteoporosis.

Osteoporosis poses significant health challenges, particularly in post-menopausal women and the elderly, leading to increased morbidity, mortality, and socioeconomic burdens. While various medications are available, all may have side their adverse effects that underscore the need for alternative interventions. Notably, omega-3 polyunsaturated fatty acids (PUFAs) have shown potential in enhancing bone strength and minimizing bone loss. However, the role of n-3 VLC-PUFAs is less explored. Surprisingly, it has now been found that n-3 VLC-PUFAs have favourable effects on bone mineralization and cellular processes, shedding light on their potential in enhancing bone health. The VLCFA compositions herein may hence be used in preventive treatment to maintain, or improve, the bone strength capacity, and to reduce the risk of osteoporosis, such as in an aging person. In one embodiment, the VLCFA compositions herein are for use by women in the menopause or post-menopausal stages.

As used herein, when referring to a subject, this term encompasses both human and non-human animal bodies, and non-human animals also include fish, such as farmed fish.

Accordingly, the invention provides compositions comprising very long chain fatty acids having a chain length of 24 carbon atoms or more, for one or more of
increasing mineralization in bone or tissue;
Increasing bone density, or bone mineral density;
improving cell proliferation and cell growth;
improving cell migration.

### Key Findings/Studies Conducted:

Investigation of the functions of n-3 VLCPUFAs in skin using in vivo Atlantic Salmon and in vitro human and fish skin models have been conducted (Examples 1 and 2 below). The purpose of the first study was to investigate the effect of dietary n-3 VLCPUFAs on the maturation and development of skin tissue in juvenile Atlantic salmon (Salmo salar) in vivo, as well as their effects on skin keratocyte and human skin fibroblast cell migration in vitro (Example 2). The fish were fed different dietary levels of n-3 VLCPUFAs, and changes in skin morphology were analysed and the effects on skin tissue fatty acid composition were determined. In vitro experiments using human dermal fibroblasts and salmon keratocytes were conducted to investigate the effect of VLCPUFAs on the migration capacity of the cells. The results demonstrated that increased dietary levels of VLCPUFAs in the fish feed led to an increased epidermis thickness and more rapid scale maturation in salmon skin in vivo, leading to a more mature skin morphology, and possibly more robust skin, at an earlier life stage. The cell culture experiments with primary keratinocytes from salmon and human dermal fibroblasts were performed to elucidate the potential functions of VLCPUFAs, particularly on cell proliferation and migration, in these cell types. The human skin fibroblasts and salmon skin keratocytes supplemented with VLCPUFAs in vitro showed more rapid migration, indicating potentially beneficial effects of VLCPUFAs in wound healing. VLCPUFAs may have beneficial effects on skin tissue development, function, and integrity. The findings of this study demonstrate uptake of VLCPUFAs from the diet into the skin tissue of salmon, with a positive effect on skin development and maturation in terms of increased epidermis thickness, mucus cells, and scale development and mineralization in vivo. Furthermore, supplementing human and fish skin cells with VLCPUFAs resulted in increased cell migration in vitro. Taken together, these results suggest that VLCPUFAs may have beneficial effects on skin tissue development, function, and integrity, which could be important for skin health, but also on scale development, i.e. affecting mineralization, in vivo.

In another study, included as Example 3, the impact of dietary n-3 VLCPUFAs on skin and bone mineral composition, bone mineral density and gene expression profiles within the skeletal systems of Atlantic salmon was investigated. The supplementation of VLCPUFAs to juvenile Atlantic salmon, feeding the fish with different levels of VLCPUFAs, showed a significant linear increase of phosphorus, calcium and magnesium in the skin and in the backbone (vertebra tissue) of the fish with increasing levels of VLCPUFAs in the feed. Furthermore, increased mineralization of scales were seen with increasing levels of VLCPUFAs in the feed, showing a more rapid maturation of the scales with increased levels of VLCPUFAs in the feed. The study demonstrates effects of VLCPUFAs on mineralization, showing that orally administered VLCPUFAs were taken up by the skin and bone tissue, providing a resulting significant increase of P, Ca and Mg taken up in skin and bone with increasing levels of VLCPUFAs in the feed. The administration and use of a VLCFA composition hence results in increased mineralisation of skin and/or bone tissue and an increased amount P, Ca or Mg is found in the tissue. As the LC-PUFA EPA and DHA levels were kept constant between all dietary groups, the correlation between increased dietary n-3 VLC-PUFAs and elevated P, Ca, and Mg content within both the skin and vertebrae, as well as increased body mass density (BMD) of salmon vertebrae, suggest an independent effect of n-3 VLC-PUFA supplementation.

To bridge the gap between animal models and the potential relevance for human skeletal health, also the effects on human foetal osteoblasts in vitro has been investigated to elucidate the translational relevance of the findings. Use of human foetal osteoblast cells allowed us to explore cellular responses to n-3 VLC-PUFA supplementation at a mechanistic level. Hence, in an in vitro cell study, included as Example 4, using human bone cells (cell line), osteoblasts have been cultured with different levels of VLCFAs, showing an increased expression of several genes relevant for particularly proliferation of cells. The results indicate that VLCFAs stimulate to faster cell migration and mineralization of human osteoblasts, and early data on proliferation and mineralization markers show interesting results, with increased bone mineralization and cell proliferation. The in vitro studies with human foetal osteoblast cells supplemented with VLC-PUFAs or DHA identified trends in the expression of key markers associated with bone development and maturation, namely BGLAP and COL1A1, in both the DHA and VLC-PUFA groups compared with the control. These trends aligned with in vivo findings, suggesting the possibility of DHA and VLC-PUFAs influencing the expression of key markers associated with bone development and maturation. In one embodiment of the invention, the use of the VLCFA composition, such as by supplementation to a subject, has a favourable influence on gene expression of osteogenic markers and cytokine expression.

In a study involving feeding rats with a feed comprising different levels of VLCPUFAs, the effect on mineral uptake and bone density (rat leg) is analysed. Initial results indicate increased bone density in a group of rats with increased VLCPUFAs in the feed.

The skin is the largest organ in all vertebrates and serves as a protective barrier, separating the body's internal milieu from the external environment. Disruption of this barrier can lead to infection, since open wounds can act as entry portals for pathogens; therefore, a strong barrier and rapid wound healing are highly important whenever this barrier is disrupted. Both mammalian and fish skin comprise three main structural compartments: epidermis, dermis, and hypodermis. There are four major cell types in the epidermis, in which the most abundant types are keratinocytes in human skin and keratocytes in fish skin. Keratinocytes and keratocytes play essential roles in defence, since they form a tight barrier to protect against the entry of foreign substances and minimise loss of water, heat, and other components. The dermis is the second layer and is composed of elastic and fibrous tissue, making it the most impenetrable layer of the skin. The major cells in the dermis are fibroblasts, and in addition to being responsible for tissue homeostasis under normal physiological conditions, fibroblasts play a critical role in wound healing. Dermal fibroblasts located at the wound edges can become myofibroblastic and are key participants in tissue repair as they provide the contractile forces that bring the wound edges together. Finally, the hypodermis predominantly comprises adipocytes (fat cells) and its main functions are fat storage and insulation.

There are many similarities between mammalian and fish skin, such as their crucial protection against infection. The similarities in their fundamental structures make comparative studies interesting. The most obvious difference between mammalian and teleost fish skin is that teleost fish skin has living cells covered with mucus in the epidermis layer of the skin, whereas human epidermis is covered in dead keratinised cells. In addition, the skin of many fish species has scales, whereas mammalian skin may have hair. The mucus secreted by mucus cells covers the epidermis and serves to protect fish from infectious pathogens. The keratocytes covering the surface of fish skin will within hours after wounding cover the wounded area with a new protective layer of cells through rapid cell migration from the surrounding wound margins. Fibroblasts also play an essential role in the regeneration of connective tissue in fish skin and comprise part of the connective tissue known as granulation tissue, which typically grows from wound borders to replace damaged tissue. Understanding fish skin is important from an aquaculture perspective, as compromised skin integrity of farmed fish has major economical and animal welfare implications. Further, the similarities in the fundamental tissue structure of mammals and fish makes fish an interesting animal model for comparative studies of skin tissue. Comparison may identify novel model systems and contribute to a better and more comprehensive understanding of the skin biology of both species, as well as the discovery of novel mechanisms. In addition, an advantage of using fish model-based studies is that the entire development of the skin can be conveniently and comprehensively investigated within a very short period.

As detailed in Examples 1 and 2, we investigated the role of VLCPUFAs in skin tissue function by analysing the fatty acid composition and skin morphology of Atlantic salmon fed different dietary levels of VLCPUFAs. Furthermore, cell culture experiments with primary keratocytes from Atlantic salmon and human dermal fibroblasts were performed to elucidate the potential functions of VLCPUFAs in vitro, particularly on cell proliferation and migration, in these cell types. In the study feeding Atlantic salmon with increasing dietary levels of VLCPUFAs, analysis of the skin phospholipids (PL) fatty acid composition greatly reflected that of the feed, demonstrating that the fatty acid composition of skin is strongly affected by the fatty acid composition of the diet. Increasing levels of the VLCPUFA concentrate in the feed led to a gradual increase in epidermis thickness. Furthermore, Von Kossa staining, a method used to visualise mineralization, revealed a darker colour of the scales with increased VLCPUFA concentration in the feed, demonstrating a more mature scale morphology in the diet group comprising most VLPUFAs. The group fed the highest concentration of VLCPUFAs, the 10% VLCPUFA group, showed strongest effects. The epidermis in the 10% VLCPUFA group was on average thicker than that of the other groups, there were also more mucus cells in the 10% group, which is most likely due to the increased thickness of the epidermis. Additionally, the 10% group had more mineralised scales compared with the lower percentage groups, which is an indication of a more rapid maturation of the scales. The observed positive effect on scale mineralization is interesting in terms of skin health and skeletal mineralization. Several studies promote fish scales as a unique and useful model in mammalian bone research, suggesting that the teleost scale is very similar to mammalian bone. In our study, fish in all the diet groups were given relatively high amounts of EPA and DHA, yet they demonstrated enhanced mineralization of scales with additional supplementation with VLCPUFAs.

Thus, based on the findings of the studies, supplementation with VLCFAs can enhance skin and bone tissue mineralization. The observed increase in epidermis thickness, mucus cell count, and more rapid scale development in the high VLCPUFA groups indicate that skin of fish in the VLCPUFA diet groups developed more rapidly, which may provide them with more robust skin earlier in development. The development of salmon skin and bone, increase in epidermis thickness and number of mucus cells, as well as mineralization, are important developmental factors and indicators of a healthy fish. The more rapid maturation of the scales adds to this, as it is part of the protective barrier of the fish. These findings could be of importance in aquaculture, since the transfer of salmon from freshwater to seawater induces stress and challenge the skin integrity, making it especially vulnerable to wounds during this phase. Furthermore, the finding that VLCPUFAs stimulate to faster cell migration and mineralization support potential beneficial use for human health. Thus, based on our findings, administration of VLCFAs to human subjects could be useful for maintaining or enhancing bone mineralization.

In vitro salmon keratocyte migration in response to 26:6 n-3: Salmon skin contains naturally low amounts of the n-3 VLCPUFA 26:6 n-3. However, levels of this fatty acid increased significantly in the skin PL fraction in fish fed diets containing VLCPUFAs, and the effect of this fatty acid on keratocyte migration was examined. The results from the in vitro Atlantic salmon keratocyte migration experiment (Example 2), in which scales were plucked from small fish to evaluate the migration of keratocytes from the scales, showed that the VLCPUFA groups showed a significantly higher percentage of scales with cell migration. It has hence been demonstrated that primary keratocytes migrated more rapidly from scales from freshwater Atlantic salmon in cell culture when supplemented with the n-3 VLCPUFA 26:6 compared with the controls. We selected 26:6 as this was one of the VLCPUFAs that increased the most in the skin tissue after feeding fish the VLCPUFA concentrate and since this VLCPUFA is not normally detected in the skin. These findings indicate that VLCPUFAs may have a stimulatory effect on the cell migration processes in fish and may therefore have a positive effect on wound closure. From our findings, supplementation with VLCPUFAs may improve skin tissue functions in Atlantic salmon, especially in the early phases of development. Further studies have been conducted to establish how this translates to human skin and bone health.

As further detailed in Example 2, a scratch assay of human dermal fibroblasts, an in vitro cell migration and wound-healing model, was performed to study whether VLCPUFA concentrates affected cell migration. The results showed that the scratch size of the VLCPUFA group (VLCPUFAs added) was approximately 10% smaller than those seen in the control. This finding demonstrates a trend of faster migration of cells to a scratch area in the VLCPUFA group. Gene expression analysis of human dermal fibroblasts harvested directly after creating the scratch showed that expression of ELOVL4, which is involved in lipid metabolism, was significantly downregulated in the DHA group compared with the VLCPUFA and control groups, whereas carnitine palmiotyltransferase 1A (CPT1A), which is involved in mitochondrial β-oxidation of fatty acids, showed significantly higher expression in the VLCPUFA group compared with the other groups. Genes involved in cell proliferation and cell growth were also affected, please see details in the Example 2 below. The scratch assay principally covers the second stage of wound healing, which is characterised by the proliferation and migration of keratinocytes and fibroblasts. When wounded or scratched, cell monolayers respond to disruption of cell-cell contact by initiating the proliferation and migration of different cell types, such as fibroblasts and keratocytes, through alterations in growth factors and cytokine expression. We observed a significant increase in the gene expression of IL8 initially after creating the scratch in both the DHA and VLCPUFA groups compared with the control group. IL8 is a chemoattractant cytokine that plays an important role in the early stage of wound healing by acting as an attractant for cell migration to the wound site, starting with the proliferative phase/recruitment and replication of cells, which are necessary for tissue regeneration. Furthermore, vascular endothelial growth factor A (VEGFA) and fibroblast growth factor 2 (FGF2) are positive regulators of angiogenesis, which occurs during the second stage of wound healing.

Accordingly, the use of a VLCPUFA composition, as disclosed herein, may one or more of stimulate to faster cell migration, improve cell proliferation and cell growth, affect the expression of genes involved in proliferation, and thereby enhance mineralization in skin tissue or bone tissue.

Based on the findings of the study of Example 3, it has been found that the skin or bone tissue mineralization of a subject may be increased considerably as a result of administration of a VLCFA composition. In the skin from Atlantic salmon fed increasing dietary levels of VLC-PUFAs, Phosphorus increased by ~28%, and Calcium and Magnesium increased by ~32% and ~19% (P=0.02), respectively, in a group fed the highest VLC-PUFA diet compared with the control group. I the vertebrae (backbone) from Atlantic salmon fed increasing dietary levels of VLC-PUFAs, Phosphorus increased by 22%, Ca increased by ~25% and Mg increased by around 14% (P=0.01) from the control group to the High VLC-PUFA group. The mineral content in total (P, Ca, and Mg) hence increased with about 20% (in bone) and 15% (skin), from the control to the high VLCPUFA group, respectively. Accordingly, in one embodiment, the concentration of P, Ca or Mg in a skin or bone tissue is increased by at least 1%, such as least 2%, such as at least 5% compared to a control wherein no VLCFA has been administered. As seen from the Examples, the total mineralisation of tissue or bone can be increased even further, following supplement of a VLCFA composition, such as in total this may be increased with at least 1%, such as least 2%, such as at least 5%, even at least 10%, 15% or 20%.

In one embodiment, the use of a VLCPUFA composition, as disclosed herein, results in increased mineral bone density. Reference is made to Example 3, showing that the mineral bone density, as g/cm³, increased from about 0.4 g/cm³ to about 0.5 g/cm³, following supplement with a high concentrate VLCFA composition, hence a significant increase of about 20%. In one embodiment, use of the VLCFA composition may result in an increase in MBD of 1%, such as 2%, such as 5%, even 10%, 15% or 20%. Accordingly, the composition may be used for enhancing bone strength or minimizing bone loss.

The discovery of these previously unknown properties of the very long chain fatty acids, and particularly of the VLCPUFAs, providing the new technical effect recited above, clearly involves a valuable and inventive contribution to the art.

### VLCFA Compositions:

Without limiting us to the discussion above, or other specific biological mechanisms, we here disclose compositions containing VLCPUFAs and other VLCFAs obtained from natural sources that can be utilised according to the invention, e.g. to increase mineralization, such as to improve mineralization in tissue or bone, improve cell migration or proliferation or improve skin tissue functions. Compositions according to the present invention can, inter alia, be manufactured based on natural oils and methods for enriching the VLCFAs according to those that are disclosed in patent application WO2016/182452 but are not limited to the starting oils and methods that are disclosed in that application. The compositions and concentrates used in the studies of the examples (Ex. 1-4) were made for the purpose of the studies, and variants of these fall within the scope of the claimed compositions.

The invention provides compositions comprising very long chain fatty acids, preferably derived from natural oils, for the use disclosed above. The compositions herein are enriched in VLCFAs, meaning the concentration of VLCFAs is higher than in the natural oils they are derived from. The term “VLCFAs” includes either of VLCPUFAs and VLCMUFAs. In one embodiment, the composition comprises at least 1%, such as at least 2% or 3%, or more preferably at least 5% by weight of VLCPUFA, such as at least 10%, such as at least 20%, such as at least 25% by weight of VLCPUFAs. Typically, such compositions may comprise more than 10%, more than 20%, more than 30%, more than 40%, more than 50%, more than 60%, or more than 70% by weight of very long chain polyunsaturated fatty acids, such as up to about 80% VLCPUFAs. The compositions for use may also comprise other VLCFAs than VLCPUFAs/VLCn3s; such as very long chain monounsaturated fatty acids (VLCMUFAs). In some embodiments, the inclusion of such VLCMUFAs is preferred. In one embodiment, the composition for use comprises at least 0.5%, such as at least 1%, such as at least 2% by weight of VLCMUFAs. In one embodiment, the compositions comprise a mixture of different VLCFAs, preferably comprising both monounsaturated and polyunsaturated very long chain fatty acids. VLCMUFAs that may be present in the compositions are selected from any one of, including but not limited to, the following group of fatty acids: C24:1 (tetraconsenoic acid (nervonic acid)), C26:1 (hexacosenoic acid), C28:1 (octacosenoic acid), C30:1 and C32:1.

The VLCPUFAs, and any other VLCFAs, of the composition have a chain length of more than 22 carbon atoms, hence of 24 carbons or longer. In one embodiment, the composition comprises at least one VLCPUFA with a chain length of 24 carbons or longer. In one embodiment, the composition comprises VLCFAs with a chain length of 26 carbons or longer. Preferably, the composition comprises a mixture of different such VLCFAs. In this regard, the VLCFAs may have chain lengths of 24, 26, 28, 30, 32, 34, 36, 38, 40 or 42 carbons. Preferably, the composition of VLCFAs comprises a mixture of at least two VLC fatty acids, and particularly with chain lengths of 24, 26, 28 and 30 carbon atoms. The VLCFA composition preferably comprises a mixture of at least two VLCPUFAs.

In particular embodiments, the composition comprises VLCPUFAs selected from any one of the, including but not limited to, 24:5n3, 24:6n3, 26:4n3, 26:5n3, 26:6n3, 28:6n3, 28:7n3, 28:8n3 fatty acids, and particularly at least a fatty acid from the group of 26:4n3, 26:5n3, 26:6n3, 28:6n3, 28:7n3, 28:8n3 fatty acids. In a preferred embodiment, the composition comprises at least one C26n3 VLCPUFA, particularly 26:6 n3. As shown in the study of Example 1, the 26:6 n3 increased the most in the skin tissue after supplement with a VLCPUFA concentrate. In one embodiment, the composition comprises the fatty acid C26:6 n3 in at least 2.0 wt %, such as at least 5.0 wt. In one embodiment, the composition comprises a C28 fatty acid, particularly the fatty acid C28:8n3. In one embodiment the composition comprises the C28:8n3 in at least 5 wt %, such as at least 8.0 wt %.

Examples of suitable compositions, which may be varied, are also included in the Examples herein.

In one embodiment, in addition to the VLCFAs, the fatty acid composition comprises one or more LCPUFA, such as one or more C20-C22 PUFAs. In certain embodiments, the fatty acid mixtures comprise at least 5% LCPUFAs by weight of the fatty acid composition, such as at least 10%, at least 20%, at least 25%, at least 30%, at least 40%, at least 50%, or at least 60% by weight of at least one LCPUFA, such as one or more C20-C22 long chain PUFAs.

In one embodiment, the VLCFAs originate from a natural oil selected from the group of fish oil, squid oil, krill oil, copepod oil and algal oil, and is in one embodiment from fish oil. In one embodiment, the VLCFAs, including the VLCPUFAs, of the composition are unmodified as compared to the oil isolated from the natural source. Hence, in one embodiment, the chain length of the VLCFAs are unmodified, and preferably, the natural VLCFAs are included in the compositions, without any steps for elongations having taken place prior to administration.

The fatty acids of the compositions, both the VLCPUFAs and other fatty acids of the compositions, can be in the form of free fatty acids, fatty acid salts, mono-, di-, triglycerides, ethyl esters, wax esters, OAHFAs, cholesteryl esters, ceramides, phospholipids or sphingomyelins, alone or in combination. In one embodiment, the fatty acids are present in the composition in the form of free fatty acids, esters, fatty acid salts, or as mono-, di-, or triglycerides.

The compositions for use may be included in different kinds of products and should be formulated according to the use. The compositions may be administered by any administration route, including but not limited to, orally, intravenously, intramuscularly, sublingually, subcutaneously, intrathecally, buccally, rectally, vaginally, ocularly, nasally, by inhalation, transdermally, and cutaneously. For oral human use, the compositions disclosed may be formulated in variable forms, such as in oral administration forms, e.g., tablets or soft or hard capsules, chewable capsules or beads, or alternatively as a fluid composition. In one embodiment, the composition is a dietary composition. As the composition may be included in different formulations dependent on the intended use, also the concentration of the VLCFAs and dosage needed is to be adapted for the intended use.

When the subject is a human, the dosage of composition will depend on several factors, including severity of the condition or disease, the subject, the composition, the formulation, type of use, and mode of administration. In one embodiment, the VLCFA composition dose is in the range from about 0.600 g to about 6.0 g. For example, in some embodiments, the total dosage of the composition ranges from about 0.8 g to about 4.0 g, from about 1.0 g to about 4.0 g, such as about 3.0 g, or from about 1.0 g to about 2.0 g. In case of using a highly concentrated VLCFA composition, with a concentration of 5 wt % VLCFAs or higher, the dose might be much lower, for example around 0.06-0.6 g. The composition may be administered in from 1 to 10 dosages, such as from 1 to 4 times a day, such as once, twice, three times, or four times per day, and further for example, once, twice or three times per day. The composition is preferably administered over a long period, such as 12-52 weeks, e.g. 24-46 weeks. An adequate level of VLCFAs is expected to be reached after 12-16 weeks, but the subject should continue the treatment to maintain this level. In one embodiment, the subject should continue to take the composition for the rest of the life.

In one embodiment, the composition is included in an animal feed, such as in fish feed, e.g. for use as exemplified herein, including administering the feed comprising the VLCFA composition to the animal.

In one embodiment, the compositions are included in food, for human consumption.

In some embodiments the composition acts as an active pharmaceutical ingredient (API), and the composition is for use as a medicament. In some embodiments, the fatty acids of the composition is present in a pharmaceutically-acceptable amount. As used herein, the term “pharmaceutically-effective amount” means an amount sufficient to treat, e.g., reduce and/or alleviate the effects, symptoms, etc., of at least one health problem in a subject in need thereof. In another embodiment, the composition according to the invention is a food supplement, nutritional supplement, dietary supplement, or a clinical nutrition comprising VLCFAs. In a related embodiment, the invention provides a composition selected from the group of Enteral Formulas for Special Medical Use, Foods for Specified Health Uses, Food for Special Medical Purposes (FSMP), Food for Special Dietary Use (FSDU), Medical Nutrition, and a Medical Food. Such a composition is particularly suited for subjects having a deficiency of certain nutrients, such as VLCFAs.

The embodiments and features described in the context of one aspect, also apply to the other aspect of the invention and it is to be understood that every embodiment of the disclosure can optionally be combined with any one or more of the other embodiments described herein. Also elements of the Examples, or elements disclosed for one aspect, also apply for other aspects, hence the details provided for the composition for use also apply for the method or use when relevant.

### EXAMPLES

### Example 1: Dietary Supplementation of Atlantic Salmon with Increasing Levels of n-3 VLCPUFAs—Effect on Maturation and Development of Skin Tissue

A 4-week in vivo feeding trial using Atlantic salmon (Salmo Breed) was conducted in freshwater tanks at Nofima's Aquaculture Research facilities at Sunndalsøra, Norway. Five different experimental diets that were produced at the Nofima Feed Technology centre in Bergen, Norway, were tested using three tanks per diet group and 100 fish per tank.

The basal diet consisted of 50% fish meal and 10% each of wheat, wheat gluten, and soy meal concentrate, in addition to minerals and vitamin mixture. The diets were isoenergetic and contained 50% protein and 20% lipid. The five different experimental diets were supplemented with increasing levels (0%-10%) of VLCPUFAs (VLC-Conc1), while levels of EPA and DHA were kept constant.

The five diets comprised: 0, 2.5, 5.0, 7.5, or 10% of the VLC-Conc1, respectively.

The VLCPUFA concentrate was made from an anchovy fish oil distillation fraction by hydrolysis, precipitation with LiOH, and distillation, ref. WO 2016/182452. Two n-3 VLCPUFA concentrates, VLC-Conc1 and VLC-Conc2, were made, as provided in Table 1.1 below.

VLC-Conc1 was used for this salmon feeding trial and the VLC-Conc2 was used for the cell culture experiments of Ex. 2.

TABLE 1.1

| | VLC-Conc1 in | VLC-Conc2 in cell |
|---|---|---|
| | salmon feed (% of | culture trials (% |
| | total fatty acid) | of total fatty acid) |
| Sum of SFAs | 4.68 | 0.00 |
| 16:1 | 1.59 | |
| 18:1 n-9 + n-7 | 7.28 | |
| 20:1 | 0.56 | |
| 22:1 | 2.12 | |
| 24:1 | 2.78 | 1.29 |
| 26:1 | 0.23 | 2.97 |
| 28:1 | 0.06 | |
| Sum of MUFAs | 14.62 | 4.26 |
| 18:2 n-6 | 0.69 | |
| 20:4 n-6 | 0.84 | |
| 22:5 n-6 | | |
| Sum of n-6 PUFAs | 1.53 | 0.00 |
| 18:3 n-3 | 0.4 | |
| 18:4 n-3 | 1.31 | |
| 20:4 n-3 | 0.58 | |
| 20:5 n-3 | 12.88 | 0 |
| 21:5 n-3 | 0.37 | |
| 22:5 n-3 | 6.72 | 0.2 |
| 22:6 n-3 | 24.68 | 0.68 |
| Sum of n-3 LC-PUFAs | 46.94 | 0.88 |
| 24:4 n-3 | 0.68 | 0.33 |
| 24:5 n-3 | 2.91 | 1.47 |
| 24:6 n-3 | 1.51 | 0.47 |
| 26:4 n-3 | 0.77 | |
| 26:5 n-3 | 1.86 | 4.34 |
| 26:6 n-3 | 2.47 | 9.56 |
| 26:7 n-3 | 0.4 | 0.95 |
| 28:4 n-3 | 0.33 | |
| 28:5 n-3 | 0.5 | 2.04 |
| 28:6 n-3 | 0.79 | 5.31 |
| 28:7 n-3 | 0.15 | 1.42 |
| 28:8 n-3 | 9.35 | 62.25 |
| Sum of C30 fatty | 0.06 | 5.03 |
| acids | | |
| Sum of n-3 VLC-PUFAs | 21.78 | 93.17 |
| Sum of VLC-FAs | 24.97 | 97.43 |
| Others† | 0.68 | |
| Total | 90.23 | 98.31 |

The amount of fish oil was reduced from 13% to 0% as the amount of VLC-Conc1 and canola oil increased in the diets. The composition of the basal diet was identical for all groups, except for the fatty acid composition of the oil coating of the basal diet, which was coated on the pellets with a vacuum coater in the final production step.

### Sampling and Sample Preparation:

Ten fish were randomly selected from each tank at three different timepoints: start of the trial (0 days); midway (18 days); and end of the trial (28 days). Standardised tissue samples of skin from the area above the lateral line and underneath the dorsal fin was taken from 10 fish and fixed in neutral buffered 10% formalin solution (Sigma-Aldrich, UK).

### Histology:

Skin samples taken at the start, midway, and final sampling (days 0, 18, and 28, respectively) were dissected and placed in tissue-embedding cassettes (Simport, Quebec, Canada). Tissue processing was performed. Paraffin-embedded tissue samples were cut into 5-μm sections using a Microtome (Leica RM 2165), mounted on polysin-coated slides (VWR, Avantor, Pennsylvania, USA), and dried overnight at 37° C. The sections were deparaffinised and rehydrated, and staining was performed using an automated special stainer (Autostainer XL Leica Biosystems, Nussloch GmbH, Germany). Paraffin sections were stained with haematoxylin eosin (H&E) and Von Kossa (Sigma Aldrich, Darmstadt, Germany). All slides were examined using a light microscope slide scanner (Leica Microsystems, Wetzlar, Germany) and manually evaluated using an Aperio Image Scope (Leica).

Statistical analysis: Data are expressed as mean±standard error of mean (SEM). For the salmon feeding trial, weights and growth data were collected from three replicate tanks with 100 individual fish in each. Growth rates were calculated based on average fish weight in each tank. Tank values were used as experimental units (n=3). Ten fish per tank, giving a total of 30 fish per dietary group, were collected for fatty acid composition analysis. Tank values were used as experimental units (n=3) and linear regression models were used to evaluate the relationship between fatty acid tissue content and n-3 VLCPUFA levels in the feed.

### Results:

### Growth Performance:

The weight of the fish increased almost twofold during the trial, from a start weight of approximately 6 g to a termination weight of around 11 g. There was no significant difference in the final weights of the fish between the diet groups.

### Fatty Acid Composition of Skin PLs:

The total fatty acid composition of the skin PL fraction from salmon fed different dietary levels of VLC-Conc1 was identified. The skin PL fatty acid composition greatly reflected that of the feed. EPA and DHA levels were balanced between the diet groups and the data showed no significant differences in the EPA (20:5 n-3) or DHA (22:6 n-3) content of the skin between the diet groups. As the inclusion level of canola oil increased in the feed, the typical fatty acids found in this oil (18:1 n-9, 18:2 n-6, and 18:3 n-3) significantly increased in the PL fraction of the skin. It is worth noting that 18:1 n-9 was also present in VLC-Conc1.

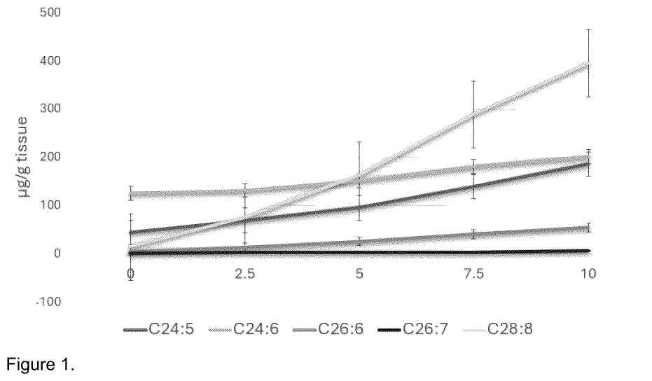

The fatty acid composition of the skin PL fraction of fish fed different levels of n-3 VLCPUFA concentrated oil (VLC-Conc1) is presented in FIG. 1, and demonstrated a clear linear increase in the n-3 VLCPUFA composition as the levels of the concentrate included in the feed. Among the different VLCPUFAs present in the concentrate, the content of 28:8 n-3 was highest, and this was also reflected in the skin. The content of the VLCPUFA 24:5 n-3 increased from a mean of around 45 μg/g tissue in the 0% group to a mean of around 186 μg/g tissue in the 10% group, while the content of 24:6 n-3 increased from approximately 125 μg/g tissue in the 0% group to 201 μg/g tissue in the 10% group. The content of 26:6 n-3 increased from approximately 5 μg/g tissue in the 0% group to approximately 56 μg/g tissue in the 10% group, whereas 26:7 n-3 and 28:8 n-3 increased from 1.4 and 14 μg/g tissue in the 0% groups, respectively, to around 5.5 and almost 400 μg/g tissue in the 10% group, respectively. Additionally, the VLC monounsaturated fatty acid (MUFA) 24:1 n-9, which was also present in VLC-Conc1, increased from a mean of around 63 μg/g tissue in the 0% group to around 80 μg/g tissue in the 10% group.

### Skin Morphology:

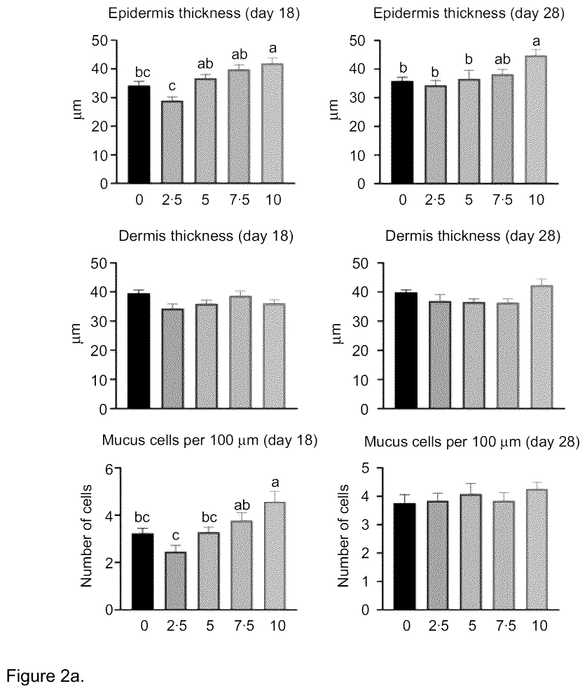

Increasing levels of the VLC-Conc1 concentrate in the feed led to a gradual increase in epidermis thickness at day 18, from 28.96±1.30 μm in the 2.5% group to 42.08±1.84 μm in the 10% group (P<0.0001), and at day 28, from 34.30±1.59 μm in the 2.5% group to 44.79±2.09 μm in the 10% group (P=0.0073). (FIG. 2(a)). Similarly, at day 18, there was a trend towards an increasing dermis thickness from the 2.5% group to the 7.5% group. At day 28, there were no significant differences in the thickness of the dermis between the groups. All groups showed increased thickness of the dermis at this point compared with that observed day 18. Furthermore, more mucus cells were detected in the higher percentage groups at day 18, with a significant difference between the groups (P=0.0002). At day 18, the 2.5% group had an average of 2.4 mucus cells per 100 μm, while the 10% group had 4.6 per 100 μm. At day 28, there were no significant differences in the number of mucus cells between the diet groups. There were no morphological changes in mucus cell distribution, indicating that the fish had satisfactory environmental conditions and were not stressed.

Von Kossa staining revealed a darker colour of the scales in the 10% diet group compared with that seen in the 0% diet group (FIG. 2(b)). This method is used to visualise mineralization; therefore, this finding demonstrates a more mature scale morphology in the 10% diet group as it showed greater mineralization. Furthermore, H&E staining showed thinner scales and more immature scales in the 0% diet group at day 18 compared with the 7.5% and 10% groups, which demonstrated thicker and more mature scales (FIG. 2(c)). Differences between the groups were most obvious between the 0% and 10% groups, and histological differences were difficult to observe in the 7.5% group. However, at day 18, immature scales were still present in the 0% group, with some in the 7.5% group but very few in the 10% group. In general, fish in the 10% group had a more mature scale structure at day 28 compared with those observed in the other groups.

### Example 2: In Vitro Cell Culture of Primary Atlantic Keratocytes and of Human Dermal Fibroblasts—Cell Migration

In these cell culture experiments fatty acids were added to growth media in the form of their sodium salts bound to bovine serum albumin (BSA; 2.7:1 molar ratio). The pH was adjusted to 7 using NH4OH. DHA was purchased from Sigma (Sigma Aldrich, Darmstadt, Germany). The n-3 VLCPUFA 26:6 n-3 and VLC-Conc2 (Table 1.1) were both prepared as 1-mM stock solutions and DHA was prepared as an 8-mM stock solution. All fatty acid solutions were stored at −80° C.

### a) Atlantic Salmon Keratocytes Study

### Atlantic Salmon Keratocytes: Cells and Media

Growth medium (GM) was prepared as L-15 (11544436, Fischer Scientific) supplemented with 5% FBS (F2442, Sigma-Aldrich), 1% anti/anti (A5955, Sigma-Aldrich; 15240062 Fischer Scientific), and 1% HEPES (H0887, Sigma-Aldrich). The test substrates added were either 25 ng/mL fibroblast growth factor (FGF) (106096-93-9, Sigma-Aldrich), 10 μM VLCPUFA (26:6 n-3), or 20 μM VLCPUFA (26:6 n-3). Controls were left untreated. FGF is known to induce cell proliferation and may be considered a positive control. The cells were incubated at 12° C. without CO₂.

Cell Migration of Primary Atlantic Salmon Keratocytes from Scales

This in vitro study using primary cell cultures of salmon scales was performed to examine whether the n-3 VLCPUFA 26:6 n-3 affected salmon keratocyte migration. In brief, seven freshwater Atlantic salmon were placed in an anaesthetic tank for approximately 5 min before being sacrificed by a blow to the head. Forceps were used to pick out single scales from the dorsal part of the lateral line of the fish and scales were placed in 12-well tissue culture plates (Thermo Fischer Scientific, Waltham, Massachusetts, USA). Each well contained 2 mL of GM with or without test substrates, which were added at the time of isolation.

The day after isolation, all wells were inspected under a microscope to determine the migration potential of the scales. All scales with migration were counted to determine the immediate effect of the VLCPUFA on cell migration. Inspection, counting, and photographing were repeated at three different time points: 24 h, 30 h, and 57 h after plucking the scales. The number of scales with cell migration was counted and divided by the number of attached scales to calculate the percentage of scales with cell migration, as attachment is a prerequisite for cell migration. The mean percentage of scales with cell migration from each fish was used in the calculations, where the individual fish was used as the experimental unit.

### b) Human Dermal Fibroblast Study

### Human Dermal Fibroblasts: Cells and Media

The ATCC PCS-201-012 commercial human dermal fibroblast cell line was cultured in low-glucose Dulbecco's modified Eagle's medium (Thermo Fischer Scientific, Waltham, Massachusetts, USA) supplemented with 10% foetal bovine serum (FBS) (F7525, Sigma-Aldrich) and 0.1% anti/anti (A5955, Sigma-Aldrich; 15240062 Fischer Scientific) in tissue culture flasks (Thermo Fischer Scientific, Waltham, Massachusetts, USA). The cells were maintained at 37° C. in a humidified atmosphere with 5% C02 and sub-cultivated when they reached confluence. Cells were used for experiments between passages 4 and 6 in this study and 3 μM VLC-Conc2 (see Table 1.1, Ex. 1) or 3 μM DHA were added as test substrates in the experiments. The control group was treated with albumin in phosphate buffer at a level comparable to that used in the VLC-Conc2 group.

### In Vitro Culture of Human Dermal Fibroblasts for Fatty Acid Composition Analysis

For fatty acid composition analysis, cells were seeded at a density of 125,000 cells/flask in 5 mL of culture media and incubated until approximately 70% confluence. Cells were then washed once in 5 mL of phosphate-buffered saline (PBS) and test substrates of either 3 μM VLC-Conc2 or 3 μM DHA were added prior to further incubation. The control group was treated with albumin in phosphate buffer at levels comparable to those of the VLC-Conc2 and DHA groups. Three replicates per substrate group were included. The following day, cells were washed once in 5 mL of PBS, fresh media containing the test substrates were added, and the flasks were further incubated overnight. The flasks were then washed twice in PBS with 1% BSA and then washed twice in PBS. Cells were loosened in 1 mL of PBS using a rubber scraper before being transferred to Eppendorf tubes and centrifuged at 550×g for 5 min.

### Scratch Assay of Human Dermal Fibroblasts, In Vitro Cell Migration, and Wound-Healing Model:

A scratch assay was performed to study whether the n-3 VLCPUFA concentrate (VLC-Conc2) affected cell migration. Cells were seeded at 1×104 cells/cm² in 8.87 cm² wells (TC Plate 6 Well, Standard, F, Sarstedt) in 3 mL of growth media. The cell migration experiment was repeated three times (n=3), with 6-9 parallels per replicate (18 for the VLC-Conc2 and DHA groups, and 21 for the control group). Test substrates were added when the cells reached approximately 70% confluency and the plates were further incubated overnight. When the cells reached approximately 90%-100% confluency, a scratch was created by pulling a pipette tip over the centre of the cell monolayer (Biosphere® Filter Tips, 70.750.211, 2-200 μL), tracing a ruler to ensure a straight line.

Culture media containing the substrates were replaced with new media after washing once with 2 mL of PBS to remove loose cells. The wells were then photographed immediately after the scratch was created and then at several timepoints (2-4-h intervals) up to 14 hours post scratching (HPS). Migration of cells into the scratched area was examined and photographed using Paula (Leica Microsystems). Cell migration was quantified as the size of the scratched area relative to the initial scratch size using the open-source image analysis software package Fiji/ImageJ (Fiji for Mac OS X).

### c) Fatty Acid Composition Analysis of Salmon Keratocytes and Human Dermal Fibroblasts:

For the fatty acid composition analysis, for both examples, total lipids were extracted from homogenised tissue or sonicated extracts of cells following the method described by Folch et al. (Folch J, Lees M, Sloane Stanley G H (1957) A simple method for the isolation and purification of total lipides from animal tissues. The Journal of biological chemistry 226, 497-509.). For skin tissue, three pooled samples of 10 individual tissue samples were analysed for lipid composition per diet group. For cells, three individual cell culture flasks per treatment group were analysed.

Ultraviolet (UV) light (366 nm) was used to detect the lipid classes, which appeared as yellow spots when placed under UV light.

The fatty acid composition of the separated lipid group was analysed using the method described by Mason and Waller (Mason M E, Waller GRJAC (1964) Dimethoxypropane induced transesterification of fats and oils in preparation of methyl esters for gas chromatographic analysis. 36, 583-586.).

The total fatty acid composition of the PL fraction from the skin tissue and total fatty acids from the cells were determined. The VLCPUFA composition of the cells was analysed using a Scion 436-GC with a split/splitless injector (splitless 1 min) with a Restek Rxi-5 ms capillary column (length, 30 m; internal diameter, 0.25 mm; and film thickness, 0.25 mM), flame ionisation detector and CompasCDS Software. Hydrogen was used as the carrier gas, with split-injection and detector temperatures of 250° C. and 270° C., respectively. The oven temperature started at 90° C. for 1.0 min and was ramped up to 200° C. at a rate of 45° C./min, then to 280° C. at a rate of 2.5° C./min, and finally to 340° C. at a rate of 10° C./min. Data were calculated by comparing the area peaks to the known amount of 23:0 IS added to the samples and presented as area percentage of the selected fatty acids.

### Gene Expression Analysis:

Fibroblasts were harvested for gene expression analysis immediately after creating the scratch (0 HPS) in the scratch assay (n=6 for all groups), and 1 day post scratching (1 DPS; n=6 for the VLC-Conc2 and DHA groups, n=9 for the control group). In brief, wells were washed once in PBS before being lysed using rNeasy lysis buffer. A sterile cell scraper with a two-position blade (Starstedt, Newton, NC) was used to loosen the cells, which were then transferred to QlAshredder spin columns, centrifuged at 550×g for 5 min, and stored at −80° C. for later analysis.

Total RNA was isolated from fibroblasts using rNeasy Plus Mini Kit (Qiagen, Hilden, Germany) according to the manufacturer's protocol. The relative gene expression level was calculated according to the DDCt method with efficiency correction (Pfaffl 2004) using RPOL2 as a reference gene (Pfaffl MWJAoqP (2004) Quantification strategies in real-time PCR. 1, 89-113.).

### Statistical Analysis:

For histology, five fish from each tank, giving a total of 15 fish per treatment, were analysed. For the salmon keratocyte migration trial, data were collected from a total of 6 individual fish in the control group, 7 in the FGF group, and 5 for each of the 10 uM VLCPUFA and 20 uM VLCPUFA groups. The individual fish were used as experimental units. For each fish, the number of scales with cell migration was divided by the number of attached scales (varying from 3-10 scales), and the mean of all wells for each fish was used in the calculations. For the human dermal fibroblast scratch assay trial, three separate trials were used, with separate thawing of the cell batch for each trial, where each test group includes 6-9 individual wells. Data are presented as mean±SEM of each trial, where n=3. Data for the human dermal fibroblasts gene expression analysis were collected from two separate scratch assay trials; one for the 0 HPS (n=6 in all groups) and another for the 1 DPS (n=6 in DHA and VLC-Conc2 groups, and n=9 in control group), where the experimental unit represents individual wells. One-way ANOVA was used to assess differences between the groups, and significant (P<0.05) differences were ranked according to Tukey's honest significant difference (HSD) test. JMP Pro 13.1.0 (SAS Institute Inc., 1989-2019) and Microsoft Office Excel software were used for the statistical analyses.

### Results:

### In Vitro Salmon Keratocyte Migration in Response to 26:6 n-3

Salmon skin contains naturally low amounts of the n-3 VLCPUFA 26:6 n-3. However, levels of this fatty acid increased significantly in the skin PL fraction in fish fed diets containing VLC-Conc1. Therefore, we examined the effect of this fatty acid on keratocyte migration. From the in vitro Atlantic salmon keratocyte migration experiment, in which scales were plucked from small fish to evaluate the migration of keratocytes from the scales, we found a significant difference between the 10 μM and 20 μM VLCPUFA (26:6 n-3) groups and FGF group compared with the control group (P=0.0145) after 24 h, where the control group had a lower migration of keratocytes from the scales. The same was seen 30 h after plucking (P=0.0295), and again, after 57 h, the VLCPUFA groups showed a significantly higher percentage of scales with cell migration (P=0.0220).

Scratch Assay and Wound-Healing of Human Dermal Fibroblasts Supplemented with n-3 VLCPUFAs

Human dermal fibroblasts were grown to approximately 70% confluence and then test substrates were added to the cells, which were incubated until they reached 100% confluence. A scratch was created, and fresh test substrates were added. The results from the scratch assay showed that the scratch size of the VLC-Conc2 group was approximately 10% smaller at 14 HPS than those seen in the control and DHA groups, albeit not significant (P=0.2597). This finding demonstrates a trend of faster migration of cells to the scratch area in the VLC-Conc2 group compared with these control groups.

Cells in each group were incubated with the relevant fatty acids for approximately 24 h prior to creating a scratch. Gene expression analysis of human dermal fibroblasts harvested directly after creating the scratch (0 HPS) showed that expression of ELOVL4, which is involved in lipid metabolism, was significantly downregulated in the DHA group compared with the VLC-Conc2 and control groups (P=0.0110), whereas carnitine palmitoyltransferase 1A (CPT1A), which is involved in mitochondrial β-oxidation of fatty acids, showed significantly higher expression in the VLC-Conc2 group compared with the other groups (P<0.0001). There were no significant differences between the groups in the expression of ceramide synthase 2 (CERS2) at this timepoint. Genes involved in cell proliferation and cell growth were also affected. Expression of transforming growth factor alpha (TGFA) was significantly different between the groups, with the highest expression seen in the DHA group (P=0.0145), and vascular endothelial growth factor A1 (VEGFA1) showed significantly higher expression in the VLC-Conc2 group compared with the other groups (P=0.0044). Additionally, fibroblast growth factor 2 (FGF2) showed significantly lower expression in both the DHA and VLC-Conc2 groups compared with the control group (P=0.0346). Genes involved in the regulation of inflammation were also affected. Interleukin 8 (IL8) showed significantly higher expression in both the DHA and VLC-Conc2 groups compared with the control group (P=0.0392).

qPCR analysis of the human dermal fibroblasts harvested at 1 DPS showed no significant difference in ELOVL4, CPT1A, or VEGFA1 expression. However, expression of CERS2, which is involved in lipid metabolism, was significantly lower in the control and VLC-Conc2 groups compared with the DHA group (P=0.0086). Genes involved in cell proliferation and cell growth were also affected at this timepoint, with significant downregulation of FGF2 in the VLC-Conc2 group compared with the DHA and control groups (P=0.0008) and significant upregulation of TGFA1 in the VLC-Conc2 group compared with the control and DHA groups (P=0.0028). Furthermore, IL8 was significantly downregulated in the VLC-Conc2 group compared with the other groups at this timepoint (P=0.0206).

There were no significant differences in the fatty acid composition in terms of long-chain fatty acids and VLCPUFAs between the treatment groups. However, there was a trend towards higher levels of the VLCPUFAs 26:3 and 26:5 n-3 in the VLC-Conc2 group. The MUFA 24:1, which was present in VLC-Conc2, also showed a trend to be higher in the VLC-Conc2 group.

### Example 3: Atlantic Salmon Feeding Trial—Effect on Skin and Bone Mineralization

The present study investigated whether dietary n-3 VLCPUFAs could increase skin and bone mineralization in Atlantic salmon (Salmo salar) in vivo. Atlantic salmon were fed different dietary levels of n-3 VLCPUFA, and changes in tissue n-3 VLC-PUFA composition, skeletal morphology, skin and bone mineral content, bone mineral density (BMD), and gene expression patterns were examined. The results demonstrated that increasing the dietary levels of n-3 VLC-PUFAs increased the mineral content of vertebrae and BMD in salmon, with subtle yet significant impacts on the expression of genes involved in bone-related processes. The findings demonstrate that n-3 VLC-PUFAs may increase bone mineralization, and thus have potential beneficial effects on bone health.

Two different n-3 VLC-PUFA concentrates were made from an anchovy fish oil distillation fraction by hydrolysis, precipitation with LiOH, and distillation. Both concentrates were produced by Epax Norway AS (Aalesund, Norway). The fatty acid composition of the concentrates used in the salmon feeding trial (this Example 3) and of the cell culture Experiments (of Example 4) are presented in Table 3.1. below Different concentrates were used in the feeding trial and cell culture experiments to single out the n-3 VLC-PUFA effect from effects of other fatty acids and increase the concentration so that smaller volumes could be added in the in vitro studies.

TABLE 3.1
Fatty acid composition of VLC-PUFA concentrates

| | VLC-PUFA | VLC-PUFA |
|---|---|---|
| | concentrate | concentrate |
| | Used in salmon feed | Used in cell culture |
| | (%) (Ex. 3) | trials (%) (Ex. 4) |
| 26:0 | 0.19 | nd |
| Sum of SFAs | 0.19 | nd |
| 22:1 n-9 | 0.33 | nd |
| 22:1 n-11 | 3.37 | nd |
| 24:1 n-9 | 2.69 | 1.29 |
| 26:1 n-9 | 0.20 | 2.97 |
| Sum of MUFAs | 6.59 | 4.26 |
| 22:5 n-6 | 0.37 | nd |
| Sum of n-6 PUFAs | 0.37 | nd |
| 18:4 n-3 | 0.20 | nd |
| 20:5 n-3 | 0.34 | nd |
| 22:5 n-3 | 4.30 | 0.20 |
| 22:6 n-3 | 13.29 | 0.68 |
| Sum of n-3 LC-PUFAs | 18.13 | 0.88 |
| 24:4 n-3 | 1.25 | 0.33 |
| 24:5 n-3 | 6.57 | 1.47 |
| 24:6 n-3 | 3.02 | 0.47 |
| 26:3 n-3 | 0.20 | nd |
| 26:4 n-3 | 1.97 | nd |
| 26:5 n-3 | 3.05 | 4.34 |
| 26:6 n-3 | 7.24 | 9.56 |
| 26:7 n-3 | 1.07 | 0.95 |
| 28:5 n-3 | 0.71 | 2.04 |
| 28:6 n-3 | 2.59 | 5.31 |
| 28:7 n-3 | 0.93 | 1.42 |
| 28:8 n-3 | 31.31 | 62.25 |
| 32:8 n-3 | 0.28 | nd |
| Sum of 30:5, 30:6, | 2.53 | 5.03 |
| 30:8 fatty acids | | |
| Sum of n-3 VLC-PUFAs | 62.72 | 93.17 |

### Feeding Trial:

Atlantic salmon ((SAL-1-22), Salmo Breed) were housed in freshwater tanks (100-L, n=100). The fish were maintained in conditions of 24 h light and temperature controlled to 12.5° C. (±0.5° C.). The water flow was set to 5 L/min and oxygen was adjusted to 85% 100% by adding oxygen to the water holding tank when needed. Mortality was recorded daily and the trial lasted 5 weeks.

### Diets:

Five different experimental diets were produced with a pellet size of 1.5 mm. Diets were made by coating (vacuum coater, Dinnisen BV, Sevenum, the Netherlands) the basal diet with different dietary levels of VLCPUFA, 0%, 2.5%, 5%, 7.5%, and 10% of total fatty acids, respectively named Control (Ctr), Low-VLC (L-VLC), Intermediate-VLC (I-VLC), Medium-High-VLC (MH-VLC), and High-VLC (H-VLC). The levels of eicosapentaenoic acid (EPA) and docosahexaenoic acid (DHA) were kept constant at 14.9% of the total fatty acids between all the groups. The amount of fish oil was reduced from 18.83% to 10.66% as the amount of VLC-PUFA oil included in the diets increased. Each diet was fed to triplicate tanks.

TABLE 3.2
Ingredient composition and PUFA content of the basal diet.

| | | | Diet | | |
|---|---|---|---|---|---|
| | | L- | groups | MH- | H- |
| | Ctr | VLC | I-VLC | VLC | VLC |
| Composition of basal | | | | | |
| mix (%) | | | | | |
| Fish meal | 50.00 | 50.00 | 50.00 | 50.00 | 50.00 |
| Soy protein concentrate | 10.00 | 10.00 | 10.00 | 10.00 | 10.00 |
| Wheat gluten | 9.00 | 9.00 | 9.00 | 9.00 | 9.00 |
| Wheat | 13.11 | 13.11 | 13.11 | 13.11 | 13.11 |
| Choline chloride | 0.50 | 0.50 | 0.50 | 0.50 | 0.50 |
| Canola lecithin | 0.50 | 0.50 | 0.50 | 0.50 | 0.50 |
| Micro ingredients | 3.01 | 3.01 | 3.01 | 3.01 | 3.01 |
| Water adjustment | −0.32 | −0.32 | −0.32 | −0.32 | −0.32 |
| Oils coated onto the | | | | | |
| basal diet mix | | | | | |
| Fish oil (Herring oil, | 13.83 | 13.04 | 12.25 | 11.45 | 10.66 |
| Nofima) | | | | | |
| VLCPUFA | | 0.80 | 1.59 | 2.39 | 3.18 |
| EPA/DHA (EPAX | | 0.09 | 0.18 | 0.27 | 0.36 |
| 6015TGN) | | | | | |
| EPA/DHA (EPAX | 0.37 | 0.28 | 0.19 | 0.09 | |
| 4060TGN) | | | | | |
| SUM | 100.00 | 100.00 | 100.00 | 100.00 | 100.00 |
| LC-PUFA and | | | | | |
| VLC-PUFA content | | | | | |
| EPA, % of diet | 1.17 | 1.17 | 1.17 | 1.17 | 1.17 |
| DHA, % of diet | 1.65 | 1.65 | 1.65 | 1.65 | 1.65 |
| EPA + DHA, % of diet | 2.82 | 2.82 | 2.82 | 2.82 | 2.82 |
| EPA + DHA, % of fatty | 14.85 | 14.85 | 14.85 | 14.85 | 14.85 |
| acids | | | | | |
| VLC-PUFA, % of diet | 0.00 | 0.47 | 0.95 | 1.42 | 1.90 |
| VLC-PUFA, % of fatty | 0.00 | 2.50 | 5.00 | 7.50 | 10.00 |
| acids | | | | | |

The weights of all the experimental fish in each tank were recorded in bulk at the start of the experiment and after 5 weeks, and growth rates were calculated based on the average weight of the fish in each tank.

### Fatty Acid Composition of Atlantic Salmon Feed

The fatty acid composition of the salmon feed is presented in Table 3.3 below. There is a linear increase in the sum of VLCPUFAs in the feed, in line with the intended fatty acid composition of the diets. Furthermore, the EPA (20:5 n-3) and DHA (22:6 n-3) content was approximately the same in all the diet groups.

TABLE 3.3
Fatty acid composition of the salmon feed.

| | Diet groups | | | | |
|---|---|---|---|---|---|
| | Ctr | L-VLC | I-VLC | MH-VLC | H-VLC |
| 14:0 | 10.74 | 9.71 | 10.54 | 9.00 | 8.84 |
| 15:0 | 0.20 | 0.19 | nd | 0.63 | 0.18 |
| 16:0 | 22.46 | 20.69 | 22.83 | 19.97 | 19.60 |
| 17:0 | 0.33 | 0.29 | 1.33 | 0.29 | 0.25 |
| 18:0 | 2.57 | 2.40 | 2.42 | 2.25 | 2.22 |
| 20:0 | 0.29 | nd | 0.26 | 0.26 | 0.25 |
| 24:0 | 0.31 | 0.31 | 0.30 | 0.34 | 0.32 |
| Sum of SFAs | 36.90 | 33.59 | 37.69 | 32.73 | 31.66 |
| 14:1 n-5 | 0.57 | 0.58 | 1.35 | 0.56 | 0.45 |
| 15:1 | 0.35 | 0.31 | 0.36 | 0.34 | 0.27 |
| 16:1 n-7 | 8.28 | 6.90 | 7.48 | 6.61 | 7.00 |
| 16:1 n-5 | 0.75 | 0.68 | 0.85 | 0.68 | 0.20 |
| 17:1 n-7 | 0.60 | 0.55 | 0.79 | 0.55 | 0.47 |
| 18:1 n-7 | 3.12 | 2.97 | 3.10 | 2.86 | 2.82 |
| 18:1 n-9 | 21.27 | 19.34 | 20.12 | 18.65 | 18.36 |
| 18:1 n-11 | 0.81 | 0.14 | 0.75 | 0.68 | 0.35 |
| 19:1 | 0.26 | 0.20 | 0.24 | 0.24 | 0.23 |
| 20:1 n-7 | 19.09 | 17.66 | 16.95 | 16.00 | 17.21 |
| 20:1 n-11 | 4.14 | 3.82 | 4.06 | 3.71 | 3.65 |
| 22:1 n-7 | 0.88 | 0.82 | 0.85 | 0.80 | 0.78 |
| 22:1 n-9 | 3.19 | 3.10 | 1.54 | 2.50 | 2.54 |
| 22:1 n-11 | 28.58 | 26.33 | 26.32 | 24.58 | 24.22 |
| 24:1 n-9 | 1.74 | 1.74 | 1.99 | 1.99 | 1.98 |
| Sum of MUFAs | 93.63 | 85.13 | 86.75 | 80.77 | 80.54 |
| 16:2 n-6 | 0.62 | 0.55 | 0.54 | 0.55 | 0.52 |
| 18:2 n-6 | 7.18 | 6.77 | 7.82 | 7.43 | 7.32 |
| 18:3 n-6 | 0.24 | 0.22 | 0.24 | 0.20 | 0.20 |
| 20:2 n-6 | 0.38 | 0.34 | 0.36 | 0.32 | 0.32 |
| 20:4 n-6 | 0.55 | 0.55 | 0.62 | 0.60 | 0.60 |
| 22:4 n-6 | 0.57 | 0.53 | 0.54 | 0.48 | 0.48 |
| Sum of n-6 PUFAs | 9.55 | 8.95 | 10.13 | 9.57 | 9.44 |
| 16:2 n-3 | 0.57 | 0.51 | 0.53 | 0.48 | 0.40 |
| 20:4 n-3 | 1.14 | 0.78 | 2.22 | 1.43 | nd |
| 20:5 n-3 | 9.84 | 9.31 | 12.44 | 10.78 | 10.51 |
| 22:5 n-3 | 1.70 | 1.79 | 2.07 | 2.18 | 2.15 |
| 22:6 n-3 | 16.45 | 15.66 | 16.16 | 15.66 | 15.41 |
| Sum of n-3 PUFAs | 31.85 | 30.01 | 35.55 | 32.49 | 30.40 |
| Sum EPA and DHA | 26.29 | 24.97 | 28.60 | 26.44 | 25.92 |
| 24:4 n-3 | 0.09 | 0.25 | 0.23 | 0.28 | 0.33 |
| 24:5 n-3 | 0.57 | 0.87 | 1.30 | 1.71 | 2.05 |
| 24:6 n-3 | 0.17 | 0.33 | 0.52 | 0.73 | 0.91 |
| 26:4 n-3 | 0.06 | 0.15 | 0.30 | 0.43 | 0.52 |
| 26:5 n-3 | 0.04 | 0.20 | 0.40 | 0.62 | 0.76 |
| 26:6 n-3 | 0.05 | 0.43 | 0.88 | 1.37 | 1.75 |
| 28:8 n-3 | 0.17 | 1.70 | 3.44 | 5.56 | 6.88 |
| Sum of n-3 VLC-PUFAs | 1.18 | 3.99 | 7.22 | 10.92 | 13.48 |
| Fat % | 21.99 | 22.46 | 22.54 | 22.50 | 22.29 |

Data are presented as mg/g feed

### Sampling and Sampling Preparation:

Whole-body samples of 20 fish from each tank were frozen at the start and end of the experiment, including 10 for whole-body lipid composition analyses and 10 for X-ray analyses. At the end of the experiment, 10 fish from each tank were randomly sampled and euthanized by an overdose of the anesthetic metacain (MS-222; 0.05 0.08 g/L). Samples of vertebrae and skin were frozen in liquid nitrogen and then stored at −80° C. for later analysis. Samples of vertebrae were stored in RNAlater (Thermo-Fisher Scientific, Waltham, Massachusetts, USA). The remaining fish in each tank were stripped for faeces, which were frozen at 20° C. for later analyses of mineral digestibility.

### Mineral Analysis

For mineral content analysis, sample preparation was performed by microwave-assisted digestion using a single reaction chamber oven (UltraWave, Milestone, Sorisole, Italy) equipped with a 22-position rack. Fine-ground sample (0.15 0.25 g) was dissolved in 1 mL of VWR Chemicals, AnalaR NORMAPUR® ACS, Reag. Ph. Eur. analytical reagent) were added. The UltraWave protocol was run using 1,500 W of microwave irradiation and a maximum temperature and pressure of 220° C. and 110 bar, respectively, and a base load of 130 mL of spectroscopy (ICP-OES) (Agilent 5110 VDV, Agilent Technologies, Mulgrave, Australia) was used to determine zinc (Zn), magnesium (Mg), phosphorous (P), and calcium (Ca) concentrations. The conditions used for mineral determination by ICP-OES were in accordance with the NS: EN 15621:2017 method adapted for OES. Mineral content analysis was performed on vertebrae and skin.

Pooled feed and faeces samples (n=10) from each tank were analysed for minerals together with the inert marker yttrium (Y), and bioavailability of minerals was determined according to Austreng E. Digestibility determination in fish using chromic oxide marking and analysis of contents from different segments of the gastrointestinal tract. Aquaculture. 769, 1978; 13(3):265-72.

### Whole-Body X-Ray and Computed Tomography (CT) Analyses of Vertebrae

### Skeletal Morphology

The morphology of the vertebral column and skull were examined by means of radiography at the beginning and end (n=5 per tank) of the experiment. Radiography was performed in a semi-digital system using a mammography X-ray source (IMS Giotto, Pontecchio Marconi, BO, Italy). X-ray images were recorded on coated photoreactive phosphorous (FCR Imaging Plates, Fujifilm, Tokyo, Japan). The image resolution was 20 pixels per mm², with exposure at 22 kV and 100 mA. Plates were read using an FCR Profect Reader (Fujifilm, Tokyo, Japan). The images were evaluated with respect to any deviation of normal morphology of skeletal structures.

### CT

The BMD measurements were done with a Skyscan 1275 micro-CT scanner (CS). The samples and phantoms were scanned with an aluminium filter and a source voltage and current at 50 kV and 200 uA, and a pixel size of 10 μm. The scans were reconstructed with nrecon-reconstruction-software (nrecon, version 1.7.3), with a beam hardening correction of 55 and the min value of the CS to image conversion 0.0000-0.08000. The BMD were found with CT-analyser (CTan, v 1.20.3.0). The scans of the phantoms (0.25 g/cm³ and 0.75 g/cm³) were used for calibration. The BMD in the samples were found by selecting which part of the scans are bones and finding the average BMD in CTan.

### Microarray

Transcriptome analyses of vertebrae (six samples per group) were performed using 15 k Atlantic salmon DNA microarray SIQ-6 (GPL30031). RNA was extracted using a Biomek 4000 robot using Agencourt RNAdvance Tissue kit (Beckman Coulter), and the quality was assessed using an Agilent Bioanalyzer 2100, RNA 6000 nano kit (RIN>8).

Microarrays, reagents, and equipment were manufactured by Agilent Technologies. RNA amplification and labelling were performed using the One-Color Quick Amp Labelling Kit, and the Gene Expression Hybridization kit was used for fragmentation of labelled RNA. After overnight hybridization (17 h, 65° C., rotation speed of 0.01 g), the arrays were washed with Gene Expression Wash Buffers 1 and 2 and scanned using an Agilent scanner. Global normalization was performed by equalizing the mean intensities of all microarrays. The individual values for each feature were divided to the mean value of all samples to produce expression ratios (ERs). The log 2-ER values were normalized with the locally weighted non-linear regression (Lowess). The difference from 0 to control was assessed by criteria: expression ratio>1.75-fold and P<0.05.

### Fatty Acid Composition Analysis:

Total lipids were extracted from homogenised tissue or sonicated extracts of cells (for Ex. 4) following the method previously described by Folch et al., For both skin tissue and whole-body analyses, pooled samples from each tank (n=10 per tank, n=30 per diet group) were analysed for lipid composition. For cells, three individual cell culture flasks per treatment group were analysed. The fatty acid composition was analysed using the method described by Mason and Waller.

Methyl esters were separated and analysed in a GC (Hewlett Packard 6890; HP) with a split injector using an SGE BPX70 capillary column (length, 60 m; internal diameter, 0.25 mm; and film thickness, 0.25 um; SGE Analytical Science) flame ionsation detector, and HP chem Station software. Helium was used as the carrier gas, and both the injector and detector temperatures were 280° C. The oven temperature started at 50° C. for 1.2 min and then increased 15 to 170° C. at a rate of 4° C./min, followed by 200° C. at a rate of 0.5° C./min, and finally 280° C. at a rate of 10° C./min. The individual fatty acid peaks were identified by comparing the retention times with validated standards; GLC reference 463 and 85 (Nu-chek Prep, Elysian, MN). The absolute amount of fatty acid per gram of tissue or cells was calculated using 23:0 methyl ester as an internal standard (IS). Unidentified peaks were not included in the total fatty acid calculation.

The VLC-PUFA composition of the diets was analysed using a Scion 436-GC with a split/splitless injector (splitless 1 min) with a Restek Rxi-5 ms capillary column (length, 30 m; internal diameter, 0.25 mm; and film thickness, 0.25 mM), flame ionisation detector and CompasCDS Software. Hydrogen was used as the carrier gas, with split-injection and detector temperatures of 250° C. and 270° C., respectively. The oven temperature started at 90° C. for 1 min and was ramped up to 200° C. at a rate of 45° C./min, then 280° C. at a rate of 2.5° C./min, and finally 340° C. at a rate of 10° C./min. Data were calculated by comparing the area peaks to the known amount of 23:0 IS added to the samples and were presented as area percentage of the selected fatty acids.

The VLC-PUFA composition of cells and whole-body extracts was analysed using a Scion TQ 8300 GC-MS/MS with a split/splitless injector (splitless 1 min) with a Restek Rxi-5 ms capillary column (length, 30 m; internal diameter, 0.25 mm; and film thickness, 0.25 mM). Hydrogen was used as the carrier gas, with split-injection and detector temperatures of 250° C. and 270° C., respectively. The oven temperature started at 90° C. for 1.5 min and was ramped up to 220° C. at a rate of 30° C./min, then 275° C. at a rate of 1.21° C./min, and finally 310° C. at a rate of 6.5° C./min. The instrument was run in MRM mode with 79.1-77.1 and 79-55.1 as transitions for VLC PUFAs and 143.1-55.1 for C23:0. The response factor for DHA (relative to C23:0) was calculated using standard solutions of known concentrations of DHA and C23:0. As no standards for VLC fatty acids were available for the VLC-PUFAs, the same response factor as for DHA was assumed and used to calculate concentrations of VLC-PUFAs.

### Results:

### Growth Analysis:

The mean body weights (BWs) of the fish at the start and end of the trial, along with the specific growth rate (SGR) and thermal growth coefficient (TGC) were measured. The BW of the fish increased threefold during the trial, from a mean start weight of 5.7 g to a mean final weight of 17 g. There were no significant differences in final BW (P=0.28), SGR (P=0.55), or TGC (and P=0.55) between the different diet groups. There were no observed mortalities in any of the dietary groups.

There were no significant differences in bioavailability of the minerals P or Mg between the different diet groups. However, Zn showed a significant difference between the groups, with a moderately lower bioavailability in the I-VLC and H-VLC diet groups compared with the control group (P=0.01).

### Whole-Body Fatty Acid Composition of Atlantic Salmon

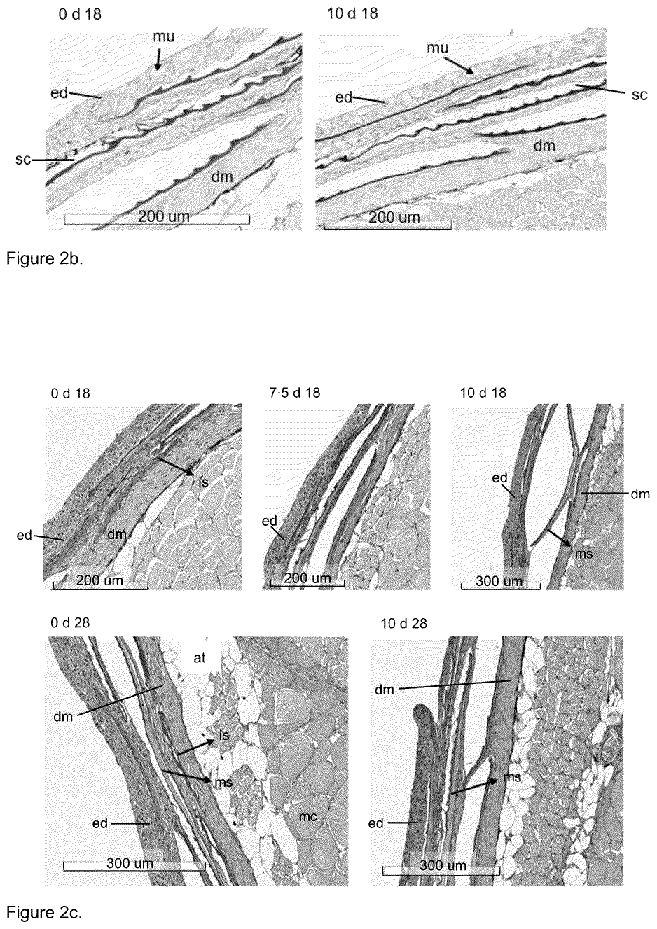

Analysis of whole-body fatty acid composition showed there were no significant differences in the amount of fatty acids containing 522 carbons, between the different dietary groups. The aim of obtaining the same whole-body content of EPA and DHA in all dietary groups was achieved, with a body content of 14 mg/g of these fatty acids. The VLCPUFA composition of whole-body homogenate showed a significant increase in VLCPUFAs from the diet, as shown in FIG. 3, with the highest amount in the High-VLC diet group (Diet group 10, with VLCPUFAs amounting to 10% of fatty acids). The 24:6 n-3 content increased from 1.07 mg/g tissue in the control group to 2.31 mg/g tissue in the High-VLC diet group (P<0.0001), and 24:5 n-3 increased from 0.86 mg/g tissue in the control group to 1.82 mg/g tissue in the High-VLC diet group (P=0.0004). While 26:7 n-3 and 26:5 n-3 were both undetected in the control group, they were detected at levels of 0.03 mg/g tissue and 0.24 mg/g tissue, respectively, in the High-VLC diet group (P<0.0001). Furthermore, 26:6 n-3 increased from 0.03 mg/g tissue in the control group to 0.88 mg/g tissue in the High-VLC diet group (P<0.0001), and 28:8 n-3 increased from 0.02 mg/g tissue in the Low-VLC diet group to 2.40 mg/g tissue in the High-VLC diet group (P<0.0001).

### Skeletal Morphology and Mineral Component Analysis of Skin and Vertebrae:

The results of the mineral component analysis of salmon skin and vertebrae are presented in Tables 3.4 and 3.5, respectively. In the skin from Atlantic salmon fed increasing dietary levels of VLC-PUFAs, Phosphorus increased by ~28% in the High VLC-PUFA group compared with the control group, and Calcium and Magnesium increased by ~32% (P=0.01) and ~19% (P=0.02), respectively.

In the vertebrae from Atlantic salmon fed increasing dietary levels of VLC-PUFAs, Phosphorus increased by 22% from the control group to the High VLC-PUFA group (P=0.02). Calcium and Magnesium also increased with increasing levels of VLC-PUFAs in the feed; Ca increased by ~25% (P=0.02) and Mg increased by around 14% (P=0.01).

TABLE 3.4
Mineral composition of Atlantic salmon skin.

| | Ctr | | L-VLC | | I-VLC | | MH-VLC | | H-VLC | | |
|---|---|---|---|---|---|---|---|---|---|---|---|
| | Mean | SEM | Mean | SEM | Mean | SEM | Mean | SEM | Mean | SEM | P-value |
| P | 10.133 | 867 | 11.000 | 577 | 11.667 | 333 | 10.667 | 333 | 13.000 | 577 | 0.02* |
| Ca | 15.333 | 1.667 | 17.000 | 1.155 | 17.667 | 882 | 17,333 | 667 | 20,333 | 882 | 0.01* |
| Mg | 453 | 33 | 467 | 17 | 490 | 15 | 487 | 23 | 540 | 32 | 0.02* |
| Zn | 47 | 6 | 51 | 1 | 51 | 2 | 49 | 4 | 56 | 6 | 0.21 |

Data are presented as mg/kg. P-value is of linear regression analysis.

TABLE 3.5
Mineral composition of Atlantic salmon vertebrae

| | Ctr | | L-VLC | | I-VLC | | MH-VLC | | H-VLC | | |
|---|---|---|---|---|---|---|---|---|---|---|---|
| | Mean | SEM | Mean | SEM | Mean | SEM | Mean | SEM | Mean | SEM | P-value |
| P | 18.000 | 0 | 18.667 | 1.333 | 18.333 | 882 | 19.000 | 1.528 | 22.000 | 0 | 0.02* |
| Ca | 32.333 | 667 | 33.000 | 3.000 | 32.667 | 1.667 | 34.333 | 32.823 | 40.333 | 333 | 0.02* |
| Mg | 603 | 7 | 607 | 30 | 620 | 15 | 627 | 20 | 690 | 6 | 0.01* |
| Zn | 140 | 26 | 127 | 15 | 140 | 12 | 123 | 13 | 173 | 26 | 0.29 |

Data are presented as mg/kg. P-value is of linear regression analysis.

### Radiography of Atlantic Salmon Vertebrae:

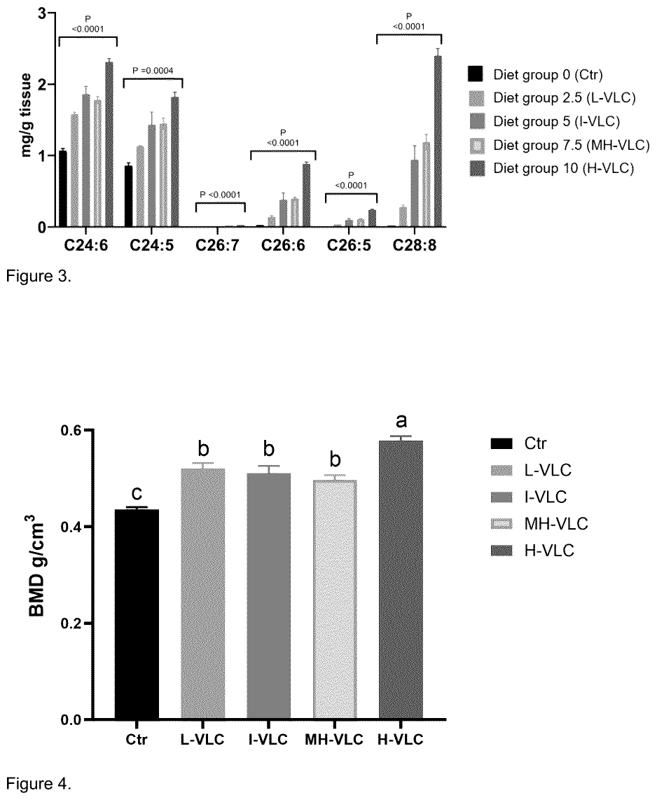

X-ray analyses of the fish in the different diet groups showed no significant differences in pathology, and the skeletal structures appeared uniform and well developed. However, CT analyses of fish from the different diet groups revealed a significantly higher bone mineral density (BMD), g/cm³, in the H-VLC-diet group compared with the other diet groups, with the lowest levels observed in the control group, see FIG. 4.

### Microarray Analysis Results

The dietary effects on gene expression in bone were small. There were two to 10 differentially expressed genes (DEGs), and only two genes showed differences in more than one group. However, several DEGs may be associated with bone-related processes. B-cell lymphoma 6a (Bcl6), a transcription factor known for its crucial role in regulating osteoblast activation and inhibition, was downregulated twofold in the High-VLC diet group. Coagulation factor IX, a gene important for normal skeletal development, was also downregulated in the same group. Furthermore, galectin-9, which is known to induce osteoblast differentiation, showed reduced expression in the Medium-High-VLC diet group. Deletion of Frizzled 9 has been shown to decrease bone mass and coincided with downregulation of isg15. Here, isg15 was significantly downregulated in three dietary groups. Atlantic salmon isg15 is a highly inducible antiviral agent, although its role in bone differentiation is unclear. However, the observed downregulation of isg15 was not associated with immune suppression since other gene members of antiviral transcription module were not affected. Upregulated mmp2 inhibitor has been shown to enhance bone formation in a rat tibial defect model and prevent bone loss in an ovariectomy-induced mouse model of osteoporosis. This gene showed the highest expression in the Low-VLC diet group. Collagenase 3 (mmp13), which is involved in restructuring the collagen matrix for bone mineralization, showed significant upregulation in both the Low-VLC and High-VLC diet groups. Osteocalcin, another gene essential for bone formation, was downregulated in the Low-VLC diet group. Procollagen-lysine dioxygenase 2, a factor influencing the formation of strong collagen fibres, was downregulated in the same group. Furthermore, retinol binding protein 4, which is associated with bone mineral density in patients with type 2 diabetes and osteopenia or osteoporosis, emerged among the selected DEGs that were downregulated in the Medium-High-VLC diet group.

### Discussion:

The present study investigated the impact of dietary n-3 VLC-PUFAs on skin and bone mineral composition, BMD, and gene expression profiles within the skeletal systems of Atlantic salmon. The choice of Atlantic salmon as an in vivo model was based on several considerations. Firstly, the physiological and genetic proximity of fish, in this case, Atlantic salmon, to humans allows for insightful extrapolation of findings relevant to our own species. Secondly, the feasibility of controlled dietary interventions and their relatively rapid growth facilitates a comprehensive assessment of the dietary impact during the developmental period. To bridge the gap between animal models and the potential relevance for human skeletal health, we also investigated the effects on human foetal osteoblasts in vitro, as detailed in Example 4 below, to elucidate the translational relevance of our findings.

The feeding trial demonstrated a substantial threefold increase in body weight of the fish, indicating successful growth during the experimental period and that supplementation of VLC-PUFAs did not exert significant effects on the overall growth performance of the fish, and X-ray analysis also showed no notable effects on the skeletal morphology. The fish in all the dietary groups had vertebrae mineral contents within the considered normal range which is expected since all the fish were fed minerals according to their requirements. The good growth and survival (100%) of the experimental fish fed increasing dietary levels of VLC-PUFAs confirmed that there were no major fish health concerns regarding this ingredient. The whole-body fatty acid composition of the salmon revealed a linear increase in several VLC-PUFAs, including 24:6 n-3, 24:5 n-3, 26:7 n-3, 26:5 n-3, 26:6 n-3, and 28:8 n-3. There were no significant differences in EPA and DHA, in accordance with the content of these fatty acids in the diet. These findings demonstrate the successful deposition of VLC-PUFAs in fish through dietary supplementation.

A significant correlation between increased dietary n-3 VLC-PUFAs and elevated P, Ca, and Mg content within both the skin and vertebrae were also found, as well as increased body mass density (BMD) of salmon vertebrae. As EPA and DHA levels were kept constant between all dietary groups, the findings suggest an independent effect of n-3 VLC-PUFA supplementation.

To investigate the possible role of intestinal absorption in the elevated mineral content, we assessed the intestinal bioavailability of the dietary minerals P, Mg, and Zn across the different dietary groups. No differences in the intestinal bioavailability of P and Mg between the dietary groups suggest that the increased mineral content in the skin and vertebrae are attributed to other functions than intestinal mineral absorption. The only mineral that showed significant differences was Zn, which was lower in the VLC-PUFA-supplemented groups. This was however not reflected in the vertebrae and skin samples, indicating that the elevated mineral levels observed in the tissues are unlikely to be driven by differences in intestinal uptake alone. The findings underscores the complexity of the interactions between fatty acids and mineral metabolism, and further suggest that n-3 VLC-PUFAs may exert their influence on mineral accumulation through distinct mechanisms that differentiate them from their more commonly studied long-chain counterparts. P and Ca are known to be the most relevant nutrients to bone health, as they comprise around 80%-90% of the mineral content of bone hydroxyapatite, while Mg depletion has been shown to have a profound effect on bone, characterized by impaired bone growth, reduced osteoblast number, and increased osteoclast number. Our observed correlation between n-3 VLC-PUFAs and mineral content further underscores the possibility that VLC-PUFAs could play a pivotal role in shaping the mineral composition of fish tissues, potentially influencing overall skeletal health.

Analysing gene expression in the vertebrae revealed subtle yet significant impacts on bone-related processes. Although the number of DEGs was relatively modest, their potential implications for bone health are noteworthy. In particular, the observed twofold downregulation of B-cell lymphoma (Bcl6) in the High-VLC diet group underscores the potential influence of n-3 VLC-PUFAs on regulatory pathways in bone, since Bcl6 is recognized for its pivotal role in orchestrating osteoblast activation and inhibition.

Coagulation factor IX, which is known to be essential for proper skeletal development, was downregulated in the vertebrae of the High-VLC diet group. Furthermore, galectin-9, which is implicated in osteoblast differentiation, displayed reduced expression in the Medium-High-VLC diet group. These regulations point to potential intricacies in the interaction between dietary factors and molecular mechanisms governing bone cell differentiation. The expression patterns further hint at complex regulatory mechanisms influenced by dietary factors. Notably, some genes critical for bone formation showed contradictory expression patterns.

### Example 4: In Vitro Cell Study, Human Foetal Osteoblasts—Effect of VLCPUFAs on Cell Proliferation, Osteogenic Differentiation, and Cytokine Expression

The present study investigated n-3 VLCPUFAs' potential effects on human osteoblast proliferation and differentiation in vitro, more specifically on cell proliferation, osteogenic differentiation, and cytokine expression. The in vitro experiments showed a potential contextual influence of n-3 VLC-PUFA supplementation on gene expression of osteogenic markers and cytokine expression.

A human foetal osteoblast cell line (hFOB 1.19, ATCC, CRL-11372) was used in the experiments. Cells were cultured according to the manufacturer's instructions. Basal medium comprised a 1:1 mixture of Ham's F12 Medium Dulbecco's Modified Eagle's Medium with 2.5 mM L-glutamine (without phenol red) (Sigma-Aldrich, Darmstadt, Germany). Complete growth medium was prepared using 0.3 mg/mL G418 (Sigma-Aldrich, Darmstadt, Germany) supplemented with 10% foetal bovine serum (T7524, Sigma-Aldrich, Darmstadt, Germany). Cells were incubated at 34° C., with 5% C02 (In-Vitro Cell NU-8600E, NUAIRE, Minnesota, U.S.) and sub-cultured at approximately 80% confluency at a ratio of 1:4. Growth medium was renewed every 2-3 days. Cells cultured for differentiation were supplemented with osteogenic factors, ascorbic acid (100 μg/mL; A8960, Sigma-Aldrich, Darmstadt, Germany) and β-glycerophosphate (10 mM; G9422, Sigma-Aldrich, Darmstadt, Germany). Once the cells reached confluency, the temperature was increased to 37° C. to stop proliferation and induce differentiation. The osteogenic media was renewed every 2-3 days.

Fatty acids were added to the growth media in the form of their sodium salts bound to bovine serum albumin (BSA, Sigma-Aldrich, Darmstadt, Germany; 2.7:1 molar ratio). The pH was adjusted to 7 using NH₄OH. n-3 VLC-PUFA (the VLC-PUFA concentrate of Table 3.1, right column) and DHA (Sigma-Aldrich, Darmstadt, Germany) concentrates were prepared as 1 and 8 mM stocks, respectively, and were added to the media at concentrations of 1 and 2 μM. All fatty acid solutions were stored at −80° C.

For gene expression analysis of cell proliferation markers, cells were plated in 6-well plates (Thermo-Fisher Scientific, Waltham, Massachusetts, USA) at passage 4 5 at a ratio of 1:3. Growth media was renewed and test substrates were added after an attachment period of 24 h. At days 2 and 3 after plating, cells were harvested for gene expression analysis. The experiment was repeated three times, with three replicates per test dose in all batches except one in which the control group had six replicates. For gene expression analysis of markers related to osteogenic differentiation, the same plating and ratio used for proliferation was used. At day 10 after inducing differentiation, the cells were supplemented with test substrates. Osteogenic growth media with and without the respective test substrates were renewed every 2 to 3 days. At day 7 after adding test substrates, cells were harvested for gene expression analysis, and growth media was frozen and stored at 80° C. for later analysis. The experiment was repeated using three separately thawed cell batches, with six replicates per test dose in one of the experiments, five replicates in the control group, four in the 1 μM and 2 μM VLC-PUFA groups, three in the 1 μM DHA group, and two in the 2 μM DHA group in another experiment, and 11 replicates in the control group and four in each of the remaining groups in the last experiment. The separate experiments were used as an experimental unit (n=3), and as there were no significant differences between the two doses within the VLC-PUFA and DHA groups, the results from the 1 and 2 μM doses were combined.

For fatty acid composition analysis, cells were plated at a ratio of 1:3 from the previous passage (passage 5) in T25 tissue culture flasks (Thermo-Fisher Scientific, Waltham, Massachusetts, USA). Osteogenic differentiation was induced, substrates were added as previously described, and cells were harvested for fatty acid composition on day 7 after adding the test substrates. Cells were washed twice in phosphate-buffered saline (PBS) with 1% BSA, then twice in PBS, and loosened in 0.5 mL of PBS by using a rubber scraper then transferred into an Eppendorf tube and centrifuged for 550×g for 5 min. The PBS was then removed, leaving only the cell pellet in the tube, which was then frozen and stored at 80° C. until further analysis.

### RNA Purification and Gene Expression Analysis

Cells were harvested for RNA extraction by washing twice in PBS, then 1 mL of QiAzol Lysis Reagent (Qiagen, Valencia, CA, USA) was added to the wells. Cells were then loosened using a rubber scraper and then transferred to a spin column and frozen at 80° C. Total RNA was isolated by adding 0.2 mL of chloroform (Merck, Germany) to the samples, vortexing for 15 s, and incubating for 2 3 min. The samples were then centrifuged at 12,000×g at 4° C. for 15 min. The aqueous phase was transferred to a new tube and one volume of 95% 100% ethanol was added. RNA was then cleaned using an RNA Clean & Concentrator −5 kit (Zymo Research, I with DNase I (Invitrogen, Carlsbad, USA). The concentration and purity of RNA were evaluated using a NanoDrop 1000 Spectrophotometer (NanoDrop Technologies, USA). cDNA was synthesised using TaqMan reverse transcriptase reagents (Applied Biosystems, Foster, City, CA, USA) according to the manufacturer's protocol.

qPCR

The expression of target genes was analysed using a QuantStudio Real-Time PCR analyser (Thermo Fisher Scientific, MA, USA) using two different assays. The proliferation genes MK167, MYBL2, CCNB1, and PCNA were analysed using PowerUp SYBR Green Master Mix (Applied Biosystems, Foster City, California, United States), whereas RUNX2, ALP, BGLAP, and COL1A1 were analysed using commercially available TaqMan probes. The specificity of the primers for the proliferation genes were confirmed by Sanger sequencing (Eurofins Genomics). The qPCR reaction mixture for the TaqMan probe assays consisted of 4 μL of a 1:10 dilution of cDNA, 0.5 μL of primer (Table 5), 0.5 μL of distilled H2O, and 5 μL of TaqMan Master Mix (TaqMan Gene expression Master Mix, Applied Biosystems 4369514), while the SYBR Green mix contained 4 μL of a 1:10 dilution of cDNA, 1 μL of forward and reverse primers (final concentration of 0.5 μM; Table 4), and 5 μL of PowerUp SYBR Green Master Mix (Applied Biosystems, Foster City, California, United States). All samples were analysed in parallel and non-template and non-enzyme controls were included. The TaqMan-based assays were performed under the following conditions: one cycle at 50° C. for 2 min; amplification for 40 cycles at 95° C. for 15 s; and 60° C. for 1 min. The other assays were performed as follows: one cycle at 50° C. for 2 min followed by 95° C. for 20 s; amplification for 40 cycles at 95° C. for 1 s followed by 60° C. for 20 s; melting at 95° C. for 1 s and 60° C. for 20 s; dissociation at 95° C. for 1 s. RefFinder (30) was used to evaluate the stability of the reference genes (GAPDH, RPOL2, EF1A, and B2M). The relative gene expression level was calculated using the ΔΔCt method (Pfaffl M W, JAoqP. Quantification strategies in real-time PCR. 2004; 1:89-113) using GAPDH as the reference gene for the proliferation genes and B2M as the reference gene for the genes related to differentiation (TaqMan probes).

### Microarray

Transcriptome analyses of vertebrae (six samples per group) were performed using 15 k Atlantic salmon DNA microarray SIQ-6 (GPL30031). RNA was extracted using a Biomek 4000 robot using Agencourt RNAdvance Tissue kit (Beckman Coulter), and the quality was assessed using an Agilent Bioanalyzer 2100, RNA 6000 nano kit (RIN>8). Microarrays, reagents, and equipment were manufactured by Agilent Technologies. RNA amplification and labelling were performed using the One-Color Quick Amp Labelling Kit, and the Gene Expression Hybridization kit was used for fragmentation of labelled RNA. After overnight hybridization (17 h, 65° C., rotation speed of 0.01 g), the arrays were washed with Gene Expression Wash Buffers 1 and 2 and scanned using an Agilent scanner. Global normalization was performed by equalizing the mean intensities of all microarrays. The individual values for each feature were divided to the mean value of all samples to produce expression ratios (ERs). The log 2-ER values were normalized with the locally weighted non-linear regression (Lowess). The difference from 0 to control was assessed by criteria: expression ratio>1.75-fold and P<0.05.

Sonicated extracts of cells were analysed as shown in Example 3.

### Cytokine Quantitation

Cell culture medium from cells harvested for osteogenic differentiation markers was collected for cytokine analysis. Cytokine levels were measured using a Bio-Plex Pro Human Cytokine 8-plex assay (#171B5018M, Bio-protocol.

### Statistical Analysis

One-way ANOVA was used to assess any differences between the treatment groups, and when P<0.05, groups were ranked according to Tukey's honest significant difference (HSD) test. Linear regression was used when appropriate to examine possible linear relationships between increasing levels of VLC-PUFA and the response variables. JMP Pro (SAS Institute Inc., 1989-2019) and Microsoft Office Excel software were used for the statistical analyses.

### Results:

In vitro cell culture of hFOB 1.19 human foetal osteoblasts

Fatty acid composition of human foetal osteoblasts

The cell culture media were supplemented with 2 μM VLC-PUFAs, and a 2 μM DHA group was included as positive control, as well as a control group without any supplementation. The fatty acid composition of the cells after 7 days of supplementation is presented in Table 4.1. Levels of EPA (20:5 n-3) were significantly different between the three groups, with a slightly higher level in the 2 μM VLC-PUFA group, although the supplemented VLC-PUFA concentrate did not contain this fatty acid (P=0.0232). DHA (22:6 n-3) was also significantly different between the groups, with the highest level observed in the 2 μM DHA group (P=0.0013), likely due to the VLC-PUFA concentrate containing this fatty acid. There was also a significant difference in the sum of n-3 PUFAs, with the highest level in the 2 μM VLC-PUFA group (P=0.0248). Levels of the VLC-PUFA 26:6 n-3 were significantly higher in the group supplemented with VLC-PUFA concentrate (2 μM) compared with the control and DHA groups (P=0.0389). The same was true also for 26:5 n-3 (P=0.304) and 28:8 n-3 (P=0.0022).

TABLE 4.1
Fatty acid composition of hFOB cells

| | | | | | | | P-value |
|---|---|---|---|---|---|---|---|
| | Control | | 2 μM DHA | | 2 μM VLC-PUFA | | (one-way |
| | Mean | SEM | Mean | SEM | Mean | SEM | ANOVA) |
| 14:0 | 37.3 | 3.02 | 38.3 | 2.23 | 41.8 | 1.79 | 0.4331 |
| 16:0 | 642.8 | 4.6 | 648.5 | 40.4 | 626.7 | 22.2 | 0.8401 |
| 17:0 | 13.4 | 0.3 | 12.5 | 0.7 | 12.5 | 0.5 | 0.4179 |
| 18:0 | 316.7 | 8.1 | 315.8 | 20.2 | 304.2 | 7.7 | 0.7698 |
| 20:0 | 11.1 | 0.2 | 10.1 | 0.6 | 10.1 | 0.4 | 0.2270 |
| 22:0 | 8.3 | 0.1 | 7.3 | 0.8 | 8.3 | 0.3 | 0.2975 |
| Sum of SFAs | 1040.6 | 0.9 | 1045.0 | 58.6 | 1019.8 | 32.3 | 0.8878 |
| 18:1 n-7 | 141.0 | 2.4 | 135.0 | 5.8 | 134.6 | 3.1 | 0.4985 |
| 18:1 n-9 | 573.7 | 10.5 | 554.6 | 19.9 | 546.5 | 14.1 | 0.4789 |
| 20:1 n-9 | 19.3 | 0.4 | 16.2 | 2.6 | 18.3 | 0.4 | 0.3994 |
| 20:1 n-11 | 5.8 | 0.4 | 7.1 | 1.1 | 7.6 | 1.5 | 0.4990 |
| 24:1 n-9 | 3.9 | 2.0 | 3.9 | 2.0 | 5.9 | 0.2 | 0.6398 |
| Sum of MUFAs | 907.7 | 12.7 | 871.5 | 30.9 | 831.9 | 45.2 | 0.3255 |
| 16:2 n-6 | 47.7 | 10.4 | 52.5 | 3.0 | 49.1 | 3.7 | 0.8640 |
| 18:2 n-6 | 41.9 | 0.8 | 40.6 | 1.5 | 41.4 | 1.2 | 0.7394 |
| 18:3 n-6 | 12.4 | 0.7 | 11.5 | 1.1 | 11.9 | 1.1 | 0.8032 |
| 20:2 n-6 | 19.0 | 0.3 | 18.8 | 0.8 | 17.3 | 0.5 | 0.1299 |
| 20:3 n-6 | 21.9 | 0.2 | 20.6 | 0.9 | 22.0 | 0.6 | 0.2916 |
| 20:4 n-6 | 78.8 | 1.3 | 106.7 | 30.3 | 76.9 | 2.0 | 0.4535 |
| 22:4 n-6 | 13.7 | 0.3 | 13.7 | 0.5 | 14.4 | 0.5 | 0.4756 |
| Sum of n-6 PUFAs | 197.6 | 18.0 | 226.2 | 42.3 | 211.0 | 7.7 | 0.7633 |
| 20:4 n-3 | 9.1 | 0.3 | 8.8 | 1.3 | 7.1 | 0.5 | 0.2897 |
| 20:5 n-3 | 18.7a | 0.4 | 19.3ab | 0.7 | 21.7b | 0.6 | 0.0232* |
| 22:5 n-3 | 27.2 | 0.4 | 26.2 | 0.8 | 26.9 | 1.5 | 0.8063 |
| 22:6 n-3 | 75.5a | 1.0 | 96.3c | 2.7 | 84.8b | 2.1 | 0.0013* |
| Sum of n-3 PUFAs | 142.3a | 3.3 | 164.0ab | 5.1 | 173.6b | 8.3 | 0.0248* |
| SUM EPA/DHA | 94.2a | 1.4 | 115.6b | 3.4 | 106.5b | 2.7 | 0.0036* |
| 24:6 n-3 | 49.3 | 4.2 | 56.5 | 1.8 | 62.2 | 7.1 | 0.2466 |
| 24:5 n-3 | 67.4 | 4.1 | 76.5 | 3.7 | 84.5 | 11.3 | 0.3161 |
| 26:6 n-3 | 2.6a | 0.2 | 3.3ab | 0.3 | 6.7b | 1.5 | 0.0389* |
| 26:5 n-3 | 1.1a | 0.2 | 1.7ab | 0.5 | 4.6b | 1.2 | 0.0304* |
| 28:8 n-3 | 0.0a | 0.0 | 0.0a | 0.0 | 11.8b | 2.6 | 0.0022* |
| SUM FA | 2635.3 | 55.8 | 2670.3 | 63.7 | 2672.8 | 73.6 | 0.9021 |
| Sum others | 180.4 | 3.9 | 168.3 | 25.8 | 186.7 | 10.1 | 0.7292 |

Data are presented as μg/g sample (n = 3)

qPCR

There was a trend towards higher expression of PCNA in the control group on day 2, which was reversed on day 3, with a trend towards higher expression in the 1 μM VLC-PUFA group and 2 μM DHA group, albeit not significant. As there were no significant differences between the two different test doses of the supplemented fatty acids, the combined effect of 1 and 2 μM VLC-PUFA as well as 1 and 2 μM DHA are presented for the cells cultured for analysis of the differentiation markers: runt-related transcription factor 2 (RUNX2), alkaline phosphatase (ALP), bone gamma-carboxyglutamate protein (BGLAP), and collagen type I alpha 1 chain (COL1A1).

There were significantly higher levels of BGLAP and COL1A1 in both the DHA and VLC-PUFA groups compared with the control group in experiment 1. However, when combining the means of all three experiments, there were no significant differences, although there was a slight trend of increased levels of BGLAP and COL1A1 in the DHA and VLC-PUFA supplemented groups.

### Cytokine Quantitation

Cytokine quantitation of growth medium from hFOB 1.19 cells cultured for gene expression analysis of differentiation markers revealed significant differences in the expression of TNF-α, IL-4, IL-2, IFN-γ, and GM-CSF, with lower expression in the 2 μM VLC-PUFA group. Expression of GM-CSF was significantly higher in the 1 μM VLC-PUFA group, while it was 1 lowest in the 2 μM VLC-PUFA group. Although not statistically significant, there was a slight trend towards lower expression of IL-8 in the two VLC-PUFA and 2 μM DHA groups compared with the control.

### Discussion:

The human foetal osteoblast cells allowed us to explore cellular responses to n-3 VLC-PUFA supplementation at a mechanistic level.

In vitro studies with human foetal osteoblast cells supplemented with VLC-PUFAs or DHA identified trends in the expression of key markers associated with bone development and maturation, namely BGLAP and COL1A1, in both the DHA and VLC-PUFA groups compared with the control. Although not statistically significant, these trends aligned with in vivo findings, suggesting the possibility of DHA and VLC-PUFAs influencing the expression of key markers associated with bone development and maturation. However, further investigations are needed to explore the underlying mechanisms and confirm these trends. As several inflammatory cytokines are known to be involved in bone metabolism, we also analysed cytokine expression in the growth medium from the human foetal osteoblast cells analysed for expression of bone differentiation markers. There was slightly lower level of IL-8 in the VLC-PUFA-supplemented groups and slightly lower level of IL-6 in the 2 μM VLC-PUFA group. Additionally, TNF-α, IL-4, IFN-γ and GM-CSF were significantly lower in the 2 μM VLC-PUFA group compared to control. These trends could indicate a subtle modulation of cytokine responses due to VLC-PUFA supplementation. As TNF-α is known to play a dual role in bone remodelling through stimulation of osteoclastogenesis and inhibition of osteoblast function, reduced levels of TNF-α can help maintain a balance between bone resorption and formation. Furthermore, IL-4 and IFN-γ have inhibitory effects on bone resorption, and reduced levels may thus also contribute to maintaining a balance between bone resorption and formation.

### CONCLUSIONS

The studies of Example 3 and 4 demonstrate for the first time that dietary supplementation with n-3 VLC-PUFAs increased the mineral content of the skin and vertebrae of Atlantic salmon and affected the expression of several genes involved in bone-related processes. CT analyses further demonstrated a significant increase in bone mineral density with increasing n-3 VLC-PUFAs in the diet, consistent with the findings of increased mineral content of the vertebrae. The human foetal osteoblast experiments showed trends towards increased expression of the genes BGLAP and COL1A1 which are known to have important functions during bone development. The presented study provides novel insight into the intricate relationship between dietary n-3 VLC-PUFAs, bone and skin mineral content, which to our knowledge, has not previously been explored. Overall, our studies contribute with valuable insight into the potential role of n-3 VLC-PUFAs in bone health. Further studies into the underlying molecular mechanisms are needed to elucidate the complex relationship between nutrition and bone physiology, particularly in relation to n-3 VLC-PUFAs, and to conclude if VLC-PUFAs have different functions in fish and mammals.

1. A method of increasing an amount of one or more of phosphorus (P), calcium (Ca) or magnesium (Mg) in skin or bone tissue of a subject, said method comprising administering to said subject a composition comprising a mixture of at least two very long chain polyunsaturated fatty acids (VLCPUFAs), wherein said VLCPUFAs have a chain length of 24 carbon atoms or more.

2. The method of claim 1, wherein said composition comprises at least 5 wt % of VLCPUFAs with a chain length of 26 or 28 carbon atoms.

3. The method of claim 1, wherein said composition further comprises at least one very long chain monounsaturated fatty acid (VLCMUFA), wherein said VLCMUFA has a chain length of at least 24 carbon atoms.

4. The method of claim 1, wherein said composition comprises one or both of fatty acids C26:6n3 and C28:8n3.

5. The method of claim 1, wherein said composition is administered to a human subject.

6. The method of claim 1, wherein said composition is administered orally.

7. The method of claim 1, wherein said subject is an animal and said composition is included in an animal feed.

8. A method of preventing or treating of bone fractures or bone fragility, said method comprising administering a very long chain fatty acid (VLCFA) composition to a subject, said composition comprising a mixture of at least two very long chain polyunsaturated fatty acids (VLCPUFAs) having a chain length of 24 carbon or more.

9. The method according to claim 8, wherein said composition comprises at least 5 wt % of VLCPUFAs with a chain length of 26 or 28 carbon atoms.

10. The method according to claim 8, wherein said method increases a bone mineral density of said subject.

11. The method of claim 8, wherein said VLCFA composition is administered dietarily to a woman in a menopause stage or a post-menopause stage.
